(19) United States
(12) Patent Application Publication
(10) Pub. No.: US 2009/0148931 A1
(43) Pub. Date: Jun. 11, 2009

(54) ILLUMINATION SYSTEMS, DEVICES, AND METHODS FOR BIOMASS PRODUCTION

(75) Inventors: Brian D. Wilkerson, Bellevue, WA (US); Wayde Watters, Kent, WA (US)

Correspondence Address: SEED INTELLECTUAL PROPERTY LAW GROUP PLLC, 701 FIFTH AVE, SUITE 5400, SEATTLE, WA, 98104, US

(73) Assignee: Bionavitas, Inc., Redmond, WA (US)

(21) Appl. No.: 12/184,164

(22) Filed: Jul. 31, 2008

Related U.S. Application Data
(60) Provisional application No. 60/953,436, filed on Aug. 1, 2007.
(60) Provisional application No. 61/061,531, filed on Jun. 13, 2008.

(51) Int. Cl. C12M 1/36; F21V 8/00; H05B 35/00; B01J 19/12; C12M 3/00

(52) U.S. Cl. 435/286.1; 362/20; 362/557; 435/292.1; 422/186; 362/555; 362/2

(57) ABSTRACT

Illumination systems, devices, and methods for cultivating biomasses. A bioreactor system is operable for growing photosynthetic organisms. The bioreactor system includes a bioreactor and an illumination system. The illumination system includes one more optical waveguides configured to light at least some of a plurality of photosynthetic organisms retained in the bioreactor. In some embodiments, the one or more optical waveguides include a plurality of structures configured to direct light energy from a solar energy collector, and a plurality of artificial light sources, along the interior of the waveguide. In some embodiments, the one more optical waveguides include a plurality of light-diffusing structures configured to guide at least a portion of the light from the solar energy collector and a plurality of artificial light sources directed along the interior of the waveguide, to the exterior of the waveguide.

### CROSS-REFERENCES TO RELATED APPLICATIONS

This application claims the benefit under 35 U.S.C. § 119(e) of U.S. Provisional Patent Application No. 60/953,436 filed Aug. 1, 2007, and U.S. Provisional Patent Application No. 61/061,531 filed Jun. 13, 2008. These two provisional applications are incorporated herein by reference in their entireties.

### TECHNICAL FIELD

The present disclosure generally relates to the field of illumination systems and, more particularly but not exclusively, to photobioreactor systems, devices, and methods using illumination systems to cultivate biomasses, photosynthetic organisms, living cells, biological active substances, or the like, or combinations thereof.

### BACKGROUND INFORMATION

Conventional electric illumination systems employing fluorescent or incandescent lamps have been used to provide light in commercial and residential settings. The fluorescent or incandescent lamps typically used, however, are not generally energy efficient or durable (long lasting).

Illumination systems have been employed in numerous applications including, for example, growing and cultivating photosynthetic organisms. Typical bioreactors used for growing, for example photosynthetic organisms, employ a constant intensity light source. One factor for cultivating biomasses (e.g., algae) in photobioreactors is providing and controlling the light necessary for the photosynthetic process. If the light intensity is too high or the exposure time too long, the growth of the algae is inhibited. Moreover, as the density of the algae cells in the bioreactor increases, algae cells closer to the light source reduce the amount of light that reaches those algae cells that are further away from the light source.

A variety of other methods and technologies exist for cultivating and harvesting biomasses such as, for example, mammalian, animal, plant, and insect cells, as well as various species of bacteria, algae, plankton, and protozoa. These methods and technologies include open-air systems and closed systems.

Algal biomasses, for example, are typically cultured in open-air systems (e.g., ponds, raceway ponds, lakes, canals, and the like) that are subject to contamination. These open-air systems are further limited by an inability to substantially control the various process parameters (e.g., temperature, incident light intensity, flow, pressure, nutrients, and the like) involved in cultivating algae.

Alternatively, biomasses are cultivated in closed systems called “bioreactors.” These closed systems allow for better control of the process parameters, but are often more costly to set up and operate. In addition, these closed systems are limited in their ability to provide sufficient light to sustain dense populations of photosynthetic organisms cultivated within.

Biomasses have many beneficial and commercial uses including, for example, uses as pollution control agents, fertilizers, food supplements, cosmetic additives, pigment additives, and energy sources, to name just a few. For example, algal biomasses are used in wastewater treatment facilities to capture fertilizers. Algal biomasses are also used to make biofuels.

Biofuels, such as biodiesel, can be used in existing diesel and compression ignition applications, where little or no modification to the engines and/or fuel delivery system is necessary. Biofuels are typically non-toxic and biodegradable; hence they provide an environmentally safe and cost-effective alternative fuel. The use of biofuels can help reduce pollution, as well as the environmental impacts of drilling, pumping, and transporting fossil-based diesel fuels.

Biofuels are already in use by some companies and governmental agencies, such as the U.S. Post Office, the Army and Air Force, the Department of Forestry, the General Services Administration, and the Agricultural Research Services. Some transit systems and school bus systems throughout the U.S. have also begun to use biofuel. Construction companies, in particular, stand to benefit tremendously from biofuel usage because most construction equipment such as, for example, cement trucks, dump trucks, bulldozers, spreaders, front loaders, cranes, backhoes, graders, and all sizes of generators is diesel-powered. In addition, biofuel can be used in other industries such as in agricultural, farming, power plants, mining, railroad, and/or marine applications.

Because of their generally non-toxic and biodegradable nature, biofuels can also be useful in marine environments for applications other than powering a diesel-powered marine engine. For example, biofuel can be used for oil spill clean-ups in the ocean and to clean the wildlife and plant life affected by those spills. Biofuels may also be useful as solvents to remove paint, or to clean out sludge from tanks used to store petroleum-based products. Further, biofuels have useful lubricant properties and can be used in a variety of machines. When used in diesel-powered engines, for example, the lubricity features of biofuels can extend the operational life of diesel-powered engines.

Commercial acceptance of illumination systems or bioreactors using biofuels is dependent on a variety of factors such as, for example, cost to manufacture, cost to operate, reliability, durability, and scalability. Commercial acceptance of bioreactors is also dependent on their ability to increase biomass production, while decreasing biomass production cost.

### BRIEF SUMMARY

In one aspect, the present disclosure is directed to an illumination system. The illumination system includes one or more optical waveguides and a plurality of light sources. In some embodiments, the one or more optical waveguides comprise one or more substantially optically transparent (light-transmitting) waveguides. In certain embodiments herein the waveguide can have any shape or form as long as it functions as an optical waveguide to direct light energy. The exemplified embodiment used throughout the application is a substantially cylindrical or cylindrical waveguide.

The illumination system may further include at least one optical fiber extending from the first end of at least one of the one or more optical waveguides, to a portion of a solar energy collector. The optical fiber is adapted to optically couple the solar energy collector to a portion of the optical waveguides (e.g., optically transparent cylindrical waveguides, and the like) and is operable to supply a first amount of light energy via the illumination system. The optical fiber may be optically coupled (directly or indirectly) to the solar energy collector. In some embodiments, the illumination system includes a plurality of light sources located proximate the first end of the optical waveguide. The plurality of light sources are operable to supply a second amount of light energy via the illumination system.

In some embodiments, a substantially optically transparent cylindrical waveguide includes a first end, a second end, an interior, and an outer surface. In some embodiments, the substantially optically transparent cylindrical waveguide may include a plurality of structures proximate the first end. In some embodiments, the plurality of structures are configured to direct the first amount of light energy from the solar energy collector and the second amount of light energy from the plurality of light sources along the interior of the substantially optically transparent cylindrical waveguide. In some embodiments, the substantially optically transparent cylindrical waveguide may further include a plurality of light-diffusing structures located along the outer surface of the substantially optically transparent cylindrical waveguide. Examples of light-diffusing structures include at least one of etchings, facets, grooves, thin-films, optical micro-prisms, lenses (e.g., micro-lenses, and the like), diffusing elements, diffractive elements (e.g., gratings, cross-gratings, and the like), texturing, and the like.

In some embodiments, the plurality of light-diffusing structures are each adapted to guide at least a portion of the first and second amounts of light energy directed along the interior of the substantially optically transparent cylindrical waveguide to the exterior of the substantially optically transparent cylindrical waveguide. The light-diffusing structures allow light energy to pass out of the waveguide.

In another aspect, the present disclosure is directed to a bioreactor system for cultivating photosynthetic organisms. The bioreactor system includes a container and an illumination assembly. The container can include an exterior surface and an interior surface. In some embodiments, the interior surface defines an isolated space configured and/or adapted to retain a plurality of photosynthetic organisms and a cultivation media.

The illumination assembly can include at least one substantially optically transparent cylindrical waveguide, a solar energy collector, a plurality of light sources, and a plurality of light-diffusing structures. In some embodiments, the illumination assembly is coupled to the container. In some embodiments, the least one substantially optically transparent cylindrical waveguide includes a first end, a second end, an interior, and an outer surface, and is received in the isolated space of the container. The solar energy collector may optically couple to a portion of the at least one substantially optically transparent cylindrical waveguide and may be adapted to supply a first amount of light energy.

In some embodiments, the plurality of light sources are located proximate the first end of the at least one substantially optically transparent cylindrical waveguide and are operable to supply a second amount of light energy. In some embodiments, a plurality of structures are proximate the first end of the at least one substantially optically transparent cylindrical waveguide and are configured to direct the first amount of light energy from the solar energy collector and the second amount of light energy from the plurality of light sources along the interior of the at least one substantially optically transparent cylindrical waveguide.

In some embodiments, one or more of the light-diffusing structures from the plurality of light-diffusing structures are located along the outer surface and are configured to guide at least a portion of the first and the second amounts of light directed along the interior of the at least one substantially optically transparent cylindrical waveguide to the exterior of the at least one substantially optically transparent cylindrical waveguide.

In yet another aspect, the present disclosure is directed to an illumination assembly including a cylindrical waveguide, at least one light source, at least one structure, and at least one light-diffusing structure.

The cylindrical waveguide includes a first end, a second end, an interior, and an outer surface. In some embodiments, the first end of the cylindrical waveguide is adapted to receive a first amount of light.

The at least one light source is located proximate the first end of the cylindrical waveguide, and is operable to supply a second amount of light energy. The at least one structure is located proximate the first end of the cylindrical waveguide, and is configured to direct the first amount of light and the second amount of light along the interior of the cylindrical waveguide.

The at least one light-diffusing structure is located along the outer surface of the cylindrical waveguide, and is configured to guide at least a portion of the first amount of light and a portion of the second amount of light directed along the interior of the cylindrical waveguide to the exterior of the cylindrical waveguide.

In some embodiments, an illumination system includes a substantially optically transparent waveguide having a first end, a second end, an interior, and an outer surface. The system further includes a solar energy collector operable to collect a first amount of light energy, a plurality of light sources, and a plurality of structures. The plurality of light sources are located proximate the first end of the waveguide and are operable to supply a second amount of light energy. The plurality of structures is proximate the first end of the waveguide and is configured to direct light energy comprising at least one of the first amount of light energy from the solar energy collector and the second amount of light energy from the plurality of light sources along the interior of the waveguide. A plurality of light-diffusing structures is located along the outer surface of the waveguide and is configured to guide at least a portion of the light energy directed along the interior of the waveguide to the exterior of the waveguide.

In some embodiments of operation, substantially all of the light energy directed along the waveguide is either light energy collected by the solar energy collector or light energy from the plurality of light sources. By way of example, when the solar energy collector is exposed to sunlight, the plurality of light sources can be OFF such that the waveguide transmits only light energy collected by the solar collector. When the solar energy collector is not exposed to sunlight or OFF, the plurality of light sources can output light energy such that the waveguide transmits only light outputted by the light sources. In some states of operation, light energy from the solar energy collector and light energy from the light sources are simultaneously delivered through the waveguide. The illumination system uses different sources of energy during a single processing sequence.

In some embodiments, an illumination system for biomass production includes a plurality of members spaced apart from one another. The members, in some embodiments, are in the form of light-diffusing elongate rods. Each elongate light-diffusing rod is adapted to receive light energy (e.g., solar light energy, non-solar light energy, or both) and to output the light energy towards the biomass. The plurality of elongate light-diffusing rods can receive light energy from a single light source or a plurality of light sources. The rods can be in the form of waveguides that are spaced evenly or unevenly from one another to achieve a desired light distribution. In some embodiments, a first set of elongate light-diffusing rods delivers light from a first light source and a second group of elongate light-diffusing rods delivers light from a second light source. Any number of additional light-diffusing rods or other optical components can be incorporated into the illumination system. In some embodiments, the light-diffusing members are in the form of plates, sheets, sheathes, and the like. For example, light can be transmitted along an edge of a sheath that is generally flat, curved, or combinations thereof. Sheathes can extend along a length of a chamber of the illumination system.

The illumination system can be incorporated into different types of biomass reactors. In some embodiments, the biomass reactor includes a biomass containment region adapted to contain the biomass in which the illumination system is at least partially disposed. The biomass containment region can include, without limitation, one or more reservoirs, tanks, and containers, as well as other structures suitable for holding a desired amount of biomass, such as a plurality of photosynthetic organisms (e.g., prokaryotic algae, eukaryotic algae, or both), cultivation media, and the like.

The illumination system, in some embodiments, further includes a solar energy delivery system adapted to receive solar energy and to direct that solar energy to light-diffusing members. In some embodiments, the solar energy delivery system includes a solar energy collector and an optical element (e.g., one or more optical fibers, optical transmission elements, etc.) that optically couples the solar energy collector to one or all of the rods. The illumination system, in some embodiments, further includes a control system that controls an amount of light energy passing through the optical element to one or more of the rods. The control system includes one or more controllers, switches, or other components for selectively controlling delivery of the light energy.

The solar energy delivery system, in some embodiments, includes an optical component for concentrating solar light energy and delivering the concentrated solar light energy to the rods. The optical component can include one or more lenses, panels, optical trains, and the like. In some embodiments, the optical component is fixedly coupled to a covering or other structure, which maintains a desired spatial relationship between the optical component and the rods. In this manner, the optical component is optically coupled to the rods via air. In some embodiments, the optical component is coupled to the rods by one or more optical connectors, such as optical fibers.

In some embodiments, the illumination system further comprises an energizable light source optically coupled to at least one of the rods. The light source is adapted to receive electrical energy and to output light energy. In some embodiments, the light source is an array of light emitting elements, such as LEDs. In some embodiments, each of the light-diffusing rods includes a first end, a second end, and an outer surface extending between the first end and the second end. An array of light emitting elements can be mounted directly to the first end of one of the rods.

In still other embodiments, an illumination system includes a covering and a reservoir containing biomass into which a plurality of light-diffusing members is at least partially submerged. At least a portion of the covering is positioned above the reservoir. In some embodiments, the covering can carry at least a portion of a solar energy delivery system optically coupled to the plurality of light-diffusing members such that energy collected by the solar energy delivery system is directed towards the members. The members then deliver the energy to the biomass for biomass production. The reservoir can be a lake, a pond, a canal, or other type of naturally occurring large body of water.

In some embodiments, an illumination system for biomass production includes a plurality of light-diffusing members, a passive light energy system, and an activatable auxiliary system. The passive light energy system is optically coupled to the plurality of light-diffusing members and receives solar light energy and delivers the solar light energy to the members. The activatable auxiliary system is also optically coupled to the plurality of light-diffusing members. The activatable auxiliary system is adapted to receive electrical energy and to generate non-solar light energy that is delivered to the plurality of elongate light-diffusing members. In some embodiments, the passive light energy system delivers solar light energy to a first group of the light-diffusing members and the activatable auxiliary system delivers light energy to a separate group of the light-diffusing members. In some embodiments, the passive light energy system and the activatable auxiliary system concurrently deliver light energy to the same light-diffusing members. When an insufficient amount of solar light energy is available (e.g., at night), the activatable auxiliary system can be used to produce a sufficient amount of non-solar light energy for biomass production. Thus, biomass production can be maintained throughout an entire day even when available solar light falls below a threshold level, for example, during the period of the day between dusk and dawn.

The illumination system, in some embodiments, further includes a controller configured to control operation of the activatable auxiliary system based, at least in part, on operation of the passive light energy system. The controller can cycle between the passive light energy system and the activatable auxiliary system. In some modes of operation, light from both the passive light energy system and the activatable auxiliary system is delivered to the members. In other modes of operation, the members only receive light energy from the passive light energy system. In yet other modes of operation, the members only receive light energy from the activatable auxiliary system. A wide range of operating states can be used to obtain the desired light delivery to the biomass in the illumination system.

In still other embodiments, a light-diffusing member includes a solar energy collector end, a terminal end, and a substantially optically transparent main body extending between the ends. The transparent main body has an outer surface such that light energy collected by the energy collector end is transmitted through the main body towards the terminal end and is emitted from the outer surface.

The energy collector end, in some embodiments, includes an integral solar energy collector. Various types of solar energy collectors may be permanently or temporarily integrated into the member. In some embodiments, a solar energy collector is embedded within material forming the main body of the member. In other embodiments, the solar energy collector is physically coupled to an external surface of the solar energy collector end.

The solar collector end, in some embodiments, extends outwardly with respect to a longitudinal axis of the member. The solar collector end, for example, may extend outwardly beyond at least a portion of or the entire outer surface of the main body. The solar collector end may have a generally v-shaped profile, u-shaped profile, spherical configuration, or flat configuration, as well as any other shape suitable for providing an enlarged feature for receiving solar energy. As such, the solar collector end can collect more solar light energy as compared to an end of a member having a substantially uniform profile along its longitudinal length.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale.

FIG. 1 is a vertical side view of an illumination assembly including a cylindrical optical waveguide according to one illustrated embodiment.

FIG. 2 is a cross-sectional view taken along the line A-A of the illumination assembly of FIG. 1 according to one illustrated embodiment.

FIG. 3 is a bottom side isometric view of an illumination assembly according to one illustrated embodiment.

FIG. 4 is a horizontal side view of an illumination assembly and a ray trace according to one illustrated embodiment.

FIG. 5 is a schematic view of an illumination system including multiple illumination assemblies according to multiple illustrated embodiments.

FIG. 6 is an exploded view of a bioreactor system including an illumination system according to one illustrated embodiment.

FIG. 7 is an exploded view of a bioreactor system including multiple illumination assemblies according to one illustrated embodiment.

FIG. 8 is a functional block diagram showing a bioreactor system according to one illustrative embodiment.

FIG. 9 is a cross-sectional side view of an open bioreactor system filled with biomass producing material according to one illustrated embodiment.

FIG. 10 is a plan view of a portable open bioreactor according to one illustrated embodiment.

FIG. 11 is a side elevational view of the bioreactor of FIG. 10 according to one illustrated embodiment.

FIG. 12 is a cross-sectional isometric view of the bioreactor of FIG. 10 taken along the line 14-14 according to one illustrated embodiment.

FIG. 13 is a cross-sectional side view of an open bioreactor system according to one illustrated embodiment.

FIG. 14 is a top front isometric view of a bioreactor system, in the form of an open air reservoir comprising a plurality of illumination assemblies, according to one illustrated embodiment.

FIG. 15 is a top front isometric view of a bioreactor system, in the form of an open air reservoir comprising a plurality of illumination assemblies, according to one illustrated embodiment.

FIG. 16 is an isometric view of an illumination system including multiple illumination assemblies according to one illustrated embodiment.

FIG. 17 is a cross-sectional side view of a modified open bioreactor system including an environment controlling structure according to one illustrated embodiment.

FIG. 18 is a cross-sectional side view of an open bioreactor system including an environment controlling structure according to one illustrated embodiment.

FIG. 19 is a top front isometric view of an environment controlling structure according to one illustrated embodiment.

FIG. 20 is a cross-sectional side view of an open bioreactor system including the environment controlling structure of FIG. 19 according to one illustrated embodiment.

FIG. 21 is a top front isometric view of a bioreactor system including a modified open air reservoir comprising a plurality environment controlling structures according to one illustrated embodiment.

FIG. 22 is a vertical side view of a light-diffusing rod partially submerged in biomass and a section of a covering according to one illustrated embodiment.

### DETAILED DESCRIPTION

In the following description, certain specific details are included to provide a thorough understanding of various disclosed embodiments. One skilled in the relevant art, however, will recognize that embodiments may be practiced without one or more of these specific details, or with other methods, components, materials, etc. In other instances, well-known structures associated with bioreactors, the transmission of effluent streams into and out of a bioreactor, the photosynthesis and lipid extraction processes of various types of biomass (e.g., algae and the like), fiber optic networks to include optical switching devices, light filters, solar collector systems to include solar array cells and solar collector mechanisms, methods of monitoring and harvesting a biomass (e.g., algae, and the like) to extract oil for biofuel purposes and/or convert a treated biomass (e.g., algae, and the like) to feedstock may not have been shown or described in detail to avoid unnecessarily obscuring the description.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment,” or “an embodiment,” or “in another embodiment,” or “in some embodiments” means that a particular referent feature, structure, or characteristic described in connection with the embodiment is included in at least one embodiment. Thus, the appearance of the phrases “in one embodiment,” or “in an embodiment,” or “in another embodiment,” or “in some embodiments” in various places throughout this specification are not necessarily all referring to the same embodiment. Furthermore, the particular features, structures, or characteristics may be combined in any suitable manner in one or more embodiments.

It should be noted that, as used in this specification and the appended claims, the singular forms “a,” “an,” and “the” include plural referents unless the content clearly dictates otherwise. Thus, for example, reference to an illumination assembly including “a cylindrical waveguide” includes a single cylindrical waveguide, or two or more cylindrical waveguides. It should also be noted that the term “or” is generally employed in its sense including “and/or” unless the content clearly dictates otherwise.

The headings provided herein are for convenience only and do not interpret the scope or meaning of the claimed invention.

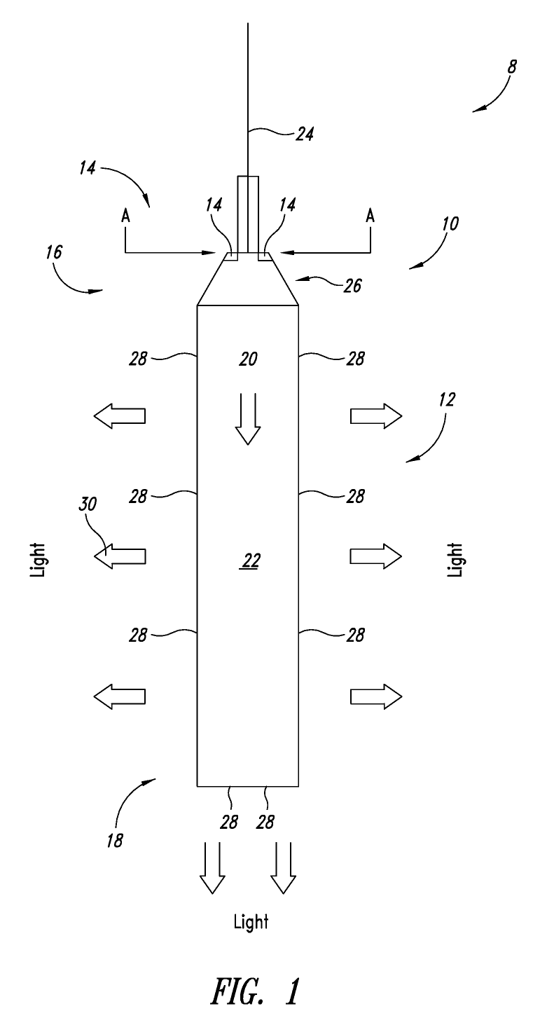

FIGS. 1-4 show examples of an illumination system 8 according to various embodiments. Referring to FIG. 1, the illumination system 8 comprises at least one illumination assembly 10 including at least one optical waveguide 12 and a plurality of light sources 14. In some embodiments, the at least one optical waveguide 12 comprises at least one optically transparent waveguide. The term “waveguide” generally refers to structures that guide waves, such as electromagnetic waves, light, sound waves, and the like. The terms “optical waveguide” or “optically transparent waveguide” generally refer to any structure having the ability to guide optical energy.

In some embodiments, the optical waveguide 12 (e.g., optically transparent waveguide, substantially optically transparent waveguide, and the like) is a light-diffusing member that includes a first end 16, a second end 18, an interior 20, and an outer surface 22. The optical waveguide 12 may take any geometric form including but not limited to, for example, cylindrical, conical, planar, regular, or irregular forms. In some embodiments, the optical waveguide 12 takes a cylindrical geometric form having a cross-section of substantially any shape including but not limited to circular, triangular, square, rectangular polygonal, and the like, as well as other symmetrical and asymmetrical shapes, or combinations thereof. In some embodiments, the optical waveguide 12 may take the form of substantially conical structures or frusto-conical structures, as well as faceted structures including but not limited to prismatoids, polyhedrons, pyramids, prisms, wedges, and the like, or combinations thereof. In some embodiments, two or more optical waveguides 12 may be coupled (optically coupled) to form, for example, an array of optical waveguides 12. In some embodiments, two or more optical waveguides 12 may be arranged so as to form a planar illumination system 8. In some embodiments, the illumination system 8 can comprise multiple optical waveguides 12 formed from a single substrate or structure. In other embodiments, the illumination system 8 can comprise multiple optical waveguides 12 forming a single substrate or structure.

In some embodiments, the optical waveguide 12 comprises at least one of a transparent, translucent, or light-transmitting material, or combinations or composites thereof. Suitable transparent, translucent, or light-transmitting materials include those materials that offer a low optical attenuation rate to the transmission or propagation of light waves. Examples of transparent, translucent, or light-transmitting materials include but are not limited to crystals, epoxies, glasses, borosilicate glasses, optically clear materials, semi-clear materials, plastics, thermo plastics, polymers, resins, thermal resins, and the like, or combinations or composites thereof.

Further examples of transparent, translucent, or light-transmitting materials include but are not limited to acetal copolymers, acrylic, acrylonitrile butadaine styrene polymers, cellulosic, diallyl phthalate, epoxies, ethylene butyl acrylate, ethylene tetrafluoroethylene, ethylene vinyl alcohol, fluorinated ethylene propylene, furan, nylon, phenolic, poly[2,2,4-trifluoro-5-trifluoromethoxy-1,3-dioxole-co-tetrafluoroethylene], poly[2,2-b]strifluoromethyl-4,5-difluoro-1,3-dioxole-co-tetrafluoroethylene], poly[2,3-(perfluoroalkenyl)perfluorotetrahydrofuran], polyacrylonitrile butadiene styrene, polybenzimidazole, polycarbonate, polyester, polyetheretherketone, polyetherimide, polyethersulfone, polyethylene, polyimide, polymethyl methacrylate, polynorbornene, polyperfluoroalkoxyethylene, polystyrene, polysulfone, polyurethane, polyvinyl chloride, polyvinylidene fluoride, thermoplastic elastomer, thermoplastic polymers, thermoplastics, thermoset polyester, thermoset polymers, transparent polymers, vinyl ester, and the like, or combinations or composites thereof. Further examples of transparent, translucent, or light-transmitting materials include but are not limited to standard optical polymer materials based on hydrocarbon (C—H) structures (e.g., polymethylmethacrylate).

In some embodiments, the transparent, translucent, or light-transmitting materials are selected such that they offer a low optical attenuation rate to the transmission or propagation of light waves in the range of about 400 nm to about 700 nm. In some embodiments, the transparent, translucent, or light-transmitting materials are selected such that they offer a low optical attenuation rate to the transmission or propagation of light waves associated with the absorption spectra of chlorophyll a and chlorophyll b. For example, the transparent, translucent, or light-transmitting materials may be selected to offer a low optical attenuation rate to the transmission or propagation of light waves in the range of about 430 nm to about 662 nm associated with the maxima of chlorophyll a and in the range of about 453 nm to about 642 nm associated with the maxima of chlorophyll b.

In some embodiments, the optical waveguide 12 comprises a substantially optically transparent cylindrical waveguide. In some embodiments, the optical waveguide 12 is an acrylic rod. In some embodiments, the illumination system 8 can comprise multiple optical waveguides 12 formed from a single substrate or structure made from, for example, at least one of a transparent, translucent, or light-transmitting material, or combinations or composites thereof. In some embodiments, the optical waveguide 12 can be made using a variety of method and techniques including but not limited to casting, solution-casting, spin-casting, injection molding, machining, micromachining, extruding, and the like, or combinations thereof.

The illumination system 8 may further include at least one optical fiber 24 extending from the first end 16 of the optical waveguide 12 to a solar energy collector. The at least one optical fiber 24 is operable to supply a first amount of light energy. In some embodiments, the illumination system 8 may further include one or more light sources 14 located proximate the first end 16 of the optical waveguide 12. In some embodiments, the one or more light sources 14 are adapted to supply a second amount of light energy. Examples of light sources 14 include, but are not limited to, artificial light sources such as, for example, electric lamps, lasers, laser diodes, LEDs, and the like, as well as natural light sources such as, for example, bioluminescence, solar radiation, radiation from astronomical objects, and the like. Further examples of light sources 14 include, but are not limited to, chemoluminescent, electroluminescent, fluorescent, incandescent, phosphorescent, or triboluminescent light sources, or combinations thereof.

At any one time, the illumination system 8 may employ natural or artificial lighting, or combinations thereof. For example, in some embodiments, the illumination system 8 may concurrently employ both artificial and natural light sources.

In some embodiments, the plurality of light sources 14 may include one or more light emitting diodes (LEDs). Suitable LEDs (including organic light-emitting diodes (OLEDs), polymer light-emitting diodes, solid-state lighting, LED lamps, and the like) come in a variety of forms and types including, for example, standard, high intensity, super bright, low current types, and the like. The “color” and/or peak emission wavelength spectrum of the emitted light generally depends on the composition and/or condition of the semi-conducting material used, and may include peak emission wavelengths in the infrared, visible, near-ultraviolet, and ultraviolet spectrum. Typically, the LEDs' color is determined by the peak wavelength of the light emitted. For example, red LEDs have a peak emission ranging from about 625 nm to about 660 nm. Examples of LED colors include but are not limited to amber, blue, red, green, white, yellow, orange-red, ultraviolet, and the like. Further examples of LEDs include bi-color LEDs, tri-color LEDs, and the like. Emission wavelength may also depend on the current delivered to the LEDs.

In some embodiments, the plurality of light sources 14 may include a plurality of LEDs. The plurality of LEDs may take the form of, for example, at least one LED array. In some embodiments, the plurality of LEDs may take the form of a plurality of two-dimensional LED arrays or at least one three-dimensional LED array. The array of LEDs may be mounted using, for example, a flip-chip arrangement. A flip-chip is one type of integrated circuit (IC) chip mounting arrangement that does not require wire bonding between chips. Thus, wires or leads that typically connect a chip/substrate having connective elements can be eliminated to reduce the profile of the illumination assembly 10.

In some embodiments, instead of wire bonding, solder beads or other elements can be positioned or deposited on chip pads such that when the chip is mounted upside-down in/on the illumination assemblies 10, electrical connections are established between conductive traces of the illumination assemblies 10 and the chip.

In some embodiments, the LEDs can be “potted” in a clear flexible medium surrounding a short length of the optical fiber 24. This short length of the optical fiber 24 may be, in some embodiments, coupled to a solar collector via one or more optical fibers.

In some embodiments, the illumination system 8 may be configured to operate in a continuous illumination mode, a pulsed illumination mode, or combinations thereof. For example, the illumination system 8 may include a waveform generator configured to generate a first driving signal operable to vary at least one of an intensity, a frequency, a pulse ratio, a pulse intensity, a pulse duration, a pulse frequency, a pulse repetition rate, a continuous waveform frequency, a continuous waveform intensity, an illumination type, an illumination supply time, an illumination duration, an illumination time increase or decrease, a an illumination interval rate, and the like, or combinations thereof, associated with the illumination system 8.

In some embodiments, the plurality of LEDs comprise a peak emission wavelength ranging from about 440 nm to about 660 nm, an on-pulse duration ranging from about 10 μs to about 10 s, and a pulse frequency ranging from about 1 μs to about 10 s. In some embodiments, the plurality of LEDs are operable to provide a first peak emission wavelength ranging from about 430 nm to about 460 nm, a second peak emission wavelength ranging from about 650 nm to about 660 nm, and optionally a third peak emission wavelength ranging from about 500 nm to about 570 nm.

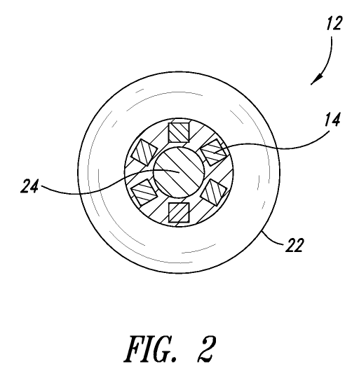

FIG. 2 shows a cross-sectional view of a site taken along the line A-A of the optical waveguide 12 of FIG. 1 according to one embodiment. As shown, at least one optical fiber 24 is received at the first end 16 of the optical waveguide 12, and six light sources 14 in the form of LED chips are arranged around the at least one optical fiber 24. In some embodiments, the LEDs can be mounted in modified T0-5 or similar standard package with, for example, a hole drilled through it.

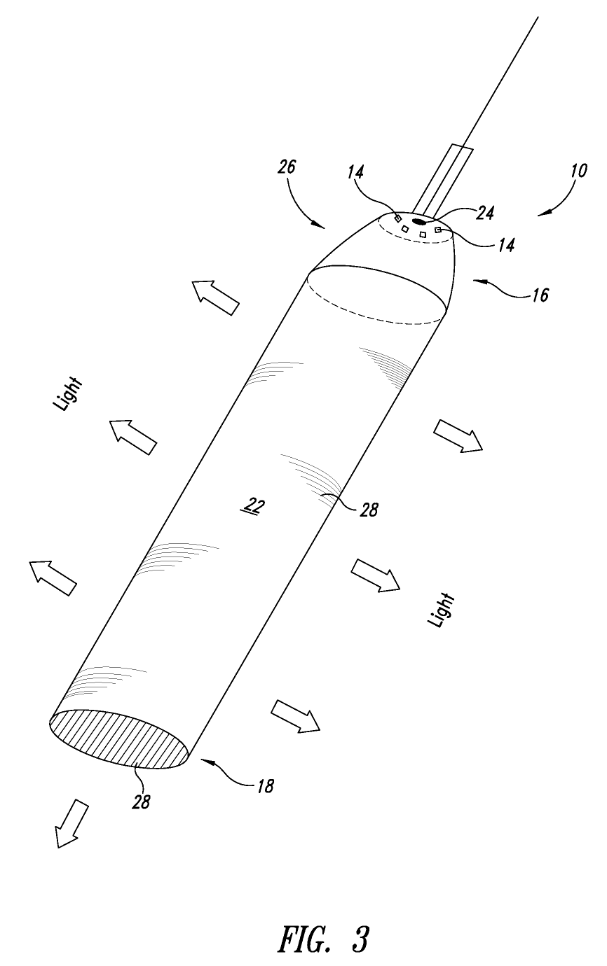

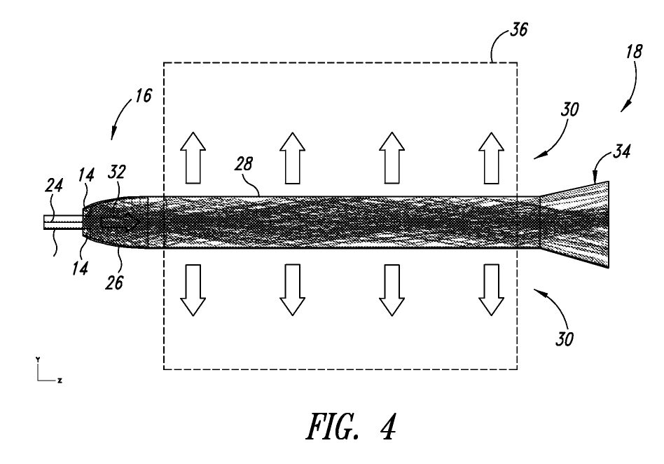

As shown in, for example, FIGS. 1 and 3, the optical waveguide 12 may further include one or more structures 26 configured to direct the first amount of light energy from a solar energy collector and the second amount of light energy from the plurality of light sources 14 along the interior 20 of the optical waveguide 12. In some embodiments, the one or more structures 26 are configured to internally reflect light (as indicated by internal reflection pattern 32 in FIG. 4) provided by the optical fiber 24 and the plurality of light sources 14. In some embodiments, the one or more structures 26 include a reflective coating, a reflective material, a mirror structure, a lens structure, or combinations thereof.

In some embodiments, the optical waveguide 12 may further include a plurality of light-diffusing structures 28 located along the outer surface 22 of the cylindrical waveguide. The plurality of light-diffusing structures 28 are configured to guide at least a portion of the first and the second amounts of light directed along the interior 20 of the optical waveguide 12 to the exterior of the optical waveguide 12 (as shown by arrows 30 in FIG. 1).

In some embodiments, the plurality of light-diffusing structures 28 are arranged such that the first and the second amounts of light directed along the interior 20 of the optical waveguide 12 are guided to the exterior to provide substantially uniform illumination 30 from a substantial portion of the surface 22 of the optical waveguide 12.

The light-diffusing structures 28 may take the form of one or more etchings, facets, grooves, thin-films, optical micro-prisms, lenses (e.g., micro-lenses, and the like), diffusing elements, diffractive elements (e.g., gratings, cross-gratings, and the like) or combinations thereof, such as represented in FIG. 3. In some embodiments, the optical waveguide 12 comprises a first refractive index, and the light-diffusing structures 28 may take the form of small particles or spheres of a second refractive index embedded within or on the surface of the optical waveguide 12. In some embodiments, the refractive index of the material of the light-diffusing structures 28 varies along the outer surface 22 of the optical waveguide 12 causing light to be refracted, diffused, or scattered at different locations along the optical waveguide 12. In such embodiments, light passing through the optical waveguide 12 and the light-diffusing structures 28 may vary along the outer surface 22 of the optical waveguide 12. In some embodiments, roughening or texturing the outer surface 22 of the optical waveguide 12 by, for example, sandblasting or machining may allow light to exit or diffuse outwardly from the optical waveguide 12. In some embodiments, machining a pattern of grooves analogous to, for example, a Fresnel lens may allow light to exit or diffuse outwardly from the optical waveguide 12.

Typically, the optimal refractive index is a function of the desired distribution of the light exiting the optical waveguide 12. Accordingly, the diffusing light pattern obtained when light passes through the light-diffusing structures 28 can be varied by changing the refractive index of the materials of the light-diffusing structures 28. In some embodiments, the light-diffusing structures 28 comprise materials having a refractive index operable to refract, scatter, or diffuse light propagated along the interior 20 of the optical waveguide 12 to the exterior of the optical waveguide 12. In some embodiments, the light-diffusing structures 28 comprise materials having a refractive index operable to substantially homogenously scatter or diffuse light propagated along the interior 20 of the optical waveguide 12 to the exterior of the optical waveguide 12.

FIG. 4 shows a non-sequential ray trace 32 of an optical fiber 24 comprising a 3 mm fiber optic shining light into the one or more structures 26 formed on the end of the optical waveguide 12 in the form of a 0.5-inch diameter solid acrylic rod according to one illustrative embodiment.

For simplicity, light rays 34 are shown coming out the end of the 3 mm fiber in a random manner. They are reflected off the inside surface of the optical waveguide 12, and remain contained within the optical waveguide 12 absent any light-diffusing structures 28. In some embodiments, however, light-diffusing structures 28 are adapted to guide optical energy within the optical waveguide 12 to the exterior to achieve, for example, a substantially uniform illumination 30 throughout a substantial portion of the surface 22 of the optical waveguide 12.

The box 36 around the optical waveguide 12 represents water, and shows that a Total Internal Reflection (TIR) is maintained in this region in the absence of the plurality of light-diffusing structures 28 located along the outer surface 22 of the cylindrical waveguide.

In some embodiments, the plurality of light-diffusing structures 28 are configured to guide light propagated within the optical waveguide 12 (as indicated by internal reflection pattern 32 in FIG. 4) to the exterior of the optical waveguide 12. In some embodiments, the plurality of light-diffusing structures 28 are configured to guide light propagated within the optical waveguide 12 (as indicated by internal reflection pattern 32 in FIG. 4) to the exterior of the optical waveguide 12 to provide substantially uniform illumination 30 throughout a substantial portion of the surface 22 of the optical waveguide 12.

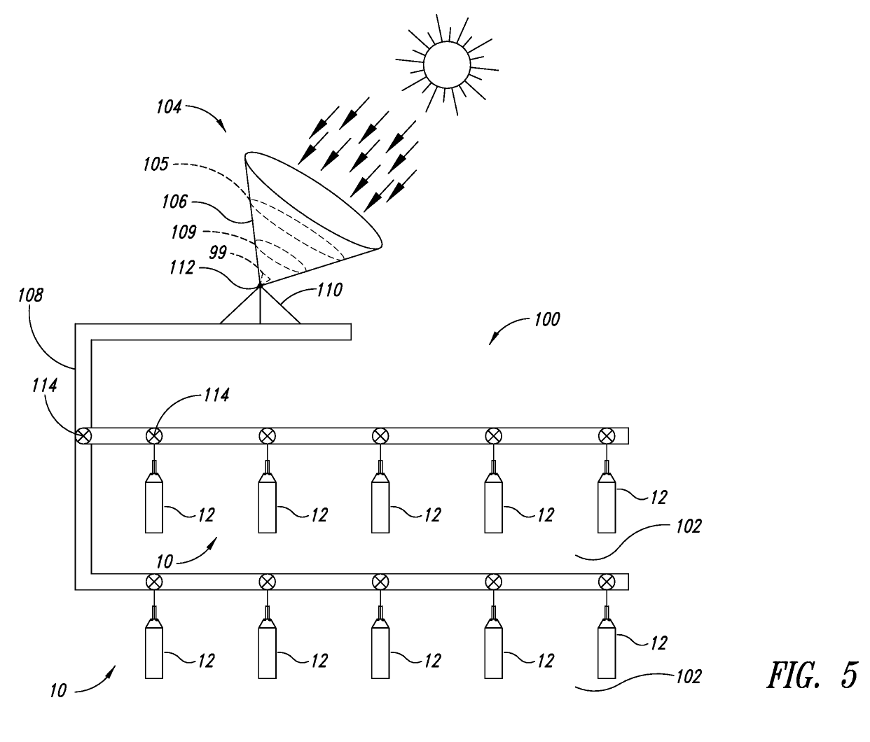

FIG. 5 shows an illumination system 100 according to one illustrated embodiment. The illumination system 100 includes one or more illumination assemblies 10 (each including at least one transparent waveguide 12) optionally coupled to a solar collector system 104 (as described in, for example, U.S. Pat. No. 5,581,447) for collecting sunlight and directing the light into the illumination system 100. In one embodiment, the solar collector system 104 is coupled via a fiber optic cable system 108 that is capable of receiving and routing sunlight into the one or more optical waveguides 12.

In some embodiments, the solar collector system 104 includes an internal transparent cover to absorb light and to reflect infrared light or alternatively, the solar collector system 104 includes a light filtering system 105 (shown schematically in dashed line) to filter out a substantial portion of the undesired wavelengths of light, such as light having wavelengths in the infrared range of wavelengths. The light filtering system 105 can include one or more covers, light filters, and the like positioned to filter out light. In some embodiments, the light filtering system 105 is located within a solar collector housing 106, which may be located on or proximate the illumination system 100, according to one embodiment. In some embodiments, the light filtering system 105 is positioned along the optical fiber 108 or positioned at another location suitable for filtering or otherwise altering light energy. An input of the light filtering system 105 may be communicatively coupled to the solar collector system 104 and an output of the light filtering system 105 can be communicatively coupled to the waveguide 108. In some embodiments, the solar collector housing 106 is located remotely from the illumination assemblies 107 but coupled to the illumination assemblies 10 via fiber optic cables or waveguides 108. The fiber optic cables or waveguides 108 are, in some instances, routed (e.g., underground) to the illumination assemblies 10.

In some embodiments, the solar collector system 104 may include a fixed portion 110 and a rotatable portion 112. The fixed portion 110 can be optically coupled to the illumination assemblies 10. The solar collector housing 106 can be rotateably coupled to the rotatable portion 112 and is controllable to be rotated, tilted, and/or swiveled (e.g., up to several degrees of freedom) so that a desired amount of solar energy can be collected. The solar collector system 104 may be combined with any of the illumination systems or bioreactors disclosed herein.

The illustrated solar collection system 104 includes an internal solar energy collector 99 (shown in dashed line in FIG. 5) for collecting solar energy. In some embodiments, the solar energy collector 99 includes one or more solar collector cells, photovoltaic cells, and the like that are arranged in a frame within the solar collector housing 106 and oriented with respect to the transparent cover to receive the light passing through the transparent cover. Different types of optical elements can form the solar energy collector 99. For example, the solar energy collector 99 may include a lens (such as a Fresnel lens) mounted to a mirrored-surface funnel-shaped collector, and may be optionally coupled to at least one fiber optic waveguide 108.

The fiber optic waveguide 108 may be bundled or independently routed to different optical waveguides 12 to selectively direct the light. In some embodiments, a portion of a light dispersion unit with a prismatic cover is coupled to an output end of the fiber optic waveguide 108 for selectively dispersing light toward a region proximate the optical waveguide 12.

Fiber optic waveguides 108 typically include a core surrounded by a cladding material, where the light propagates through the core. The core is typically made from transparent silica (e.g., glass) or a polymeric material (e.g., plastic). In one embodiment, the fiber optic waveguide 108 is made from a molecularly engineered electro-optic polymer that is commercially available from Lumera Corporation.

A control system 114 can be used to direct the light through the fiber optic waveguides 108 by selectively controlling a number of optical switches 114 arranged in the fiber optic network. The fiber optic switches 114 generally operate to re-direct, to guide, and/or to block light traveling through the fiber optic network.

Optical switches can be generally classified into the following example and non-exhaustive categories: (1) opto-mechanical switches, which include a micro-electrical mechanical system (MEMS) switches; (2) thermo-optical switches; (3) liquid-crystal and liquid-crystals-in-polymer switches; (4) gel/oil-based “bubble” switches; (5) electro-holographic switches; and others switches such as acousto-optic switches; semiconductor optical amplifiers (SOA); and ferromagnetic switches. The structure and operation of these optical switches are described in, for example, Amy Dugan et al., The Optical Switching Spectrum: A Primer on Wavelength Switching Technologies, Telecomm. Mag.; and Roland Lenz, Introduction to All Optical Switching Technologies, v. 1, (Jan. 30, 2003).

In some embodiments, the optical switches to be used with the solar collector system 104 may be adapted to operate according to any of the aforementioned principals or may be adapted to operate according to different principals. In one embodiment, the optical switch is an “Electroabsorption (EA) Optical Switch” developed by OKI® Optical Components Company. In another embodiment, the optical switch is an “Efficient Linearized Semiconductor Optical Switch” (ELSOM) developed by TRW, Inc. In yet another embodiment, the optical switch is a “Lithium Niobate (LiNbO₃) Optical Switch” developed by the Microelectronics Group of Lucent Technologies, Inc. In still yet another embodiment, the optical switch is a discrete, electro-optical switch developed by Lumera Corporation. The optical switches can include amplifiers or regenerators to condition the light, electrical signal, and/or optical signal.

The control system 114 provides control signals to cause at least some of the fiber optic waveguides 108 to emit light at successively discrete times (e.g., scan the light over an area of algae) and/or emit light at varying intensities. It is understood that at least in one embodiment, and at any discrete moment in time, at least one fiber optic waveguide 108 can be in a light-emitting state while another fiber optic waveguide 108 is in a non-light-emitting state. The control system 114 can be programmed to achieve a desired emission sequence of the light onto at least various portions of illumination regions proximate the optical waveguide 12.

In some embodiments, the illumination system 100 includes a plurality of optical waveguides 12, each in the form of a substantially optically transparent cylindrical waveguide having a first end 16, a second end 18, an interior 20, and an outer surface 22. In some embodiments, at least one optical fiber 108 extends from the first end 16 of each substantially optically transparent cylindrical waveguide 12 to the solar collector system 104. The at least one optical fiber 108 is adapted to supply a first amount of light energy to each of the substantially optically transparent cylindrical waveguides 12. In some embodiments, the plurality of light sources 14 are located proximate the first end 16 of each of the substantially optically transparent cylindrical waveguides 12, and are operable to supply a second amount of light energy.

In some embodiments, the illumination system 100 may further include a plurality of structures 26 proximate each first end 16 of the substantially optically transparent cylindrical waveguides 12. The plurality of structures 26 are configured to direct the first amount of light energy from the solar collector system 104 and the second amount of light from the plurality of light sources 14 along the interior of each of the substantially optically transparent cylindrical waveguides 12.

In some embodiments, the illumination system 100 may further include a plurality of light-diffusing structures 28 located along the outer surface 22 of each of the cylindrical waveguides, the plurality of light-diffusing structures 28 being configured to guide at least a portion of the first and the second amounts of light directed along the interior of the cylindrical waveguide to the exterior of the cylindrical waveguide.

In some embodiments, any of the described illumination systems or combinations thereof may be incorporated into a bioreactor system for cultivating photosynthetic organisms.

The term “bioreactor” as used herein and in the claims generally refers to any system, device, or structure capable of supporting a biologically active environment. Examples of bioreactors include but are not limited to fermentors, photobioreactors, stir-tank reactors, airlift reactors, pneumatically mixed reactors, fluidized bed reactors, fixed-film reactors, hollow-fiber reactors, rotary cell culture reactors, packed-bed reactors, macro and micro bioreactors, and the like, or combinations thereof.

In some embodiments, the term bioreactor refers to a device or system for growing cells or tissues in the context of cell culture, such as the disposable chamber or bag, called a CELLBAG®, made by Panacea Solutions, Inc. and usable with systems developed by Wave Biotechs, LLC. In a further embodiment, the bioreactor can be a specially designed landfill for rapidly growing, transforming, and/or degrading organic structures. In yet a further embodiment, the bioreactor comprises a sphere and a mirror located outside of the sphere, wherein the shape of the sphere maximizes a surface-to-volume ratio of the algae contained therein and a waveguide for providing light from a light source, such as sunlight, into the sphere. Further examples of bioreactors include but are not limited to open-air systems such as ponds, raceway ponds, lakes, natural reservoirs, canals, and the like, as well as regular and irregular shaped structures capable of sustaining biomass growth.

Accordingly, a bioreactor may be a closed or open system, but in certain embodiments includes any of the light sources or any of the lightning systems, devices, or methods described herein. In some embodiments, two or more bioreactors may be coupled (e.g., physically coupled, fluidically coupled, optically coupled, or the like) together to form a multi-reactor system. In further embodiments, the two or more bioreactors may be coupled in parallel in series, or combinations thereof.

The term “biomass” as used herein and in the claims generally refers to any biological material. Examples of a “biomass” include but are not limited to photosynthetic organisms, living cells, biological active substances, plant matter, living, and/or recently living biological materials, and the like. Further examples of a “biomass” include but are not limited to mammalian, animal, plant, and insect cells, as well as various species of bacteria, algae, plankton, and protozoa.

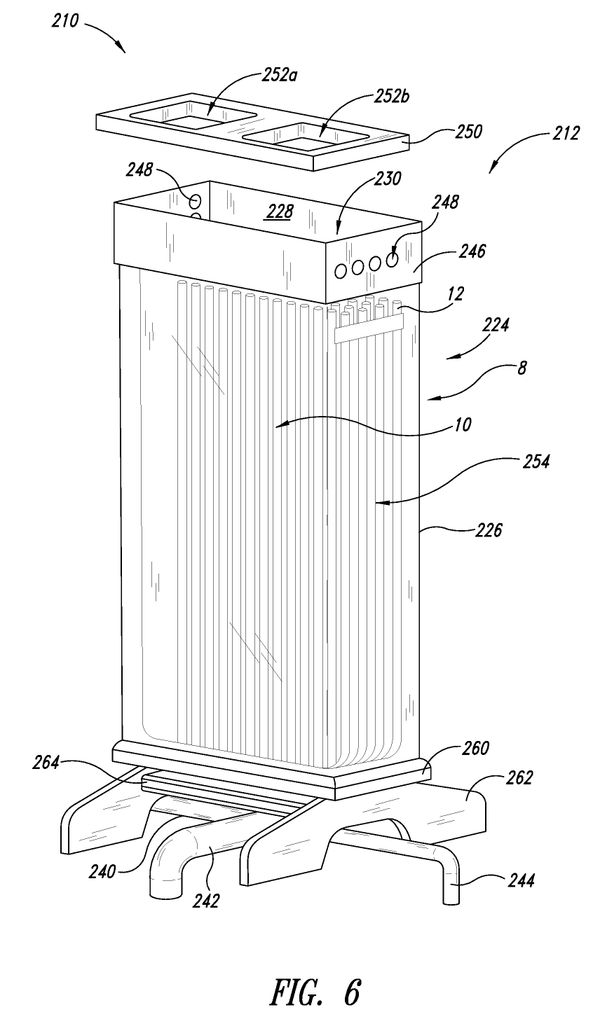

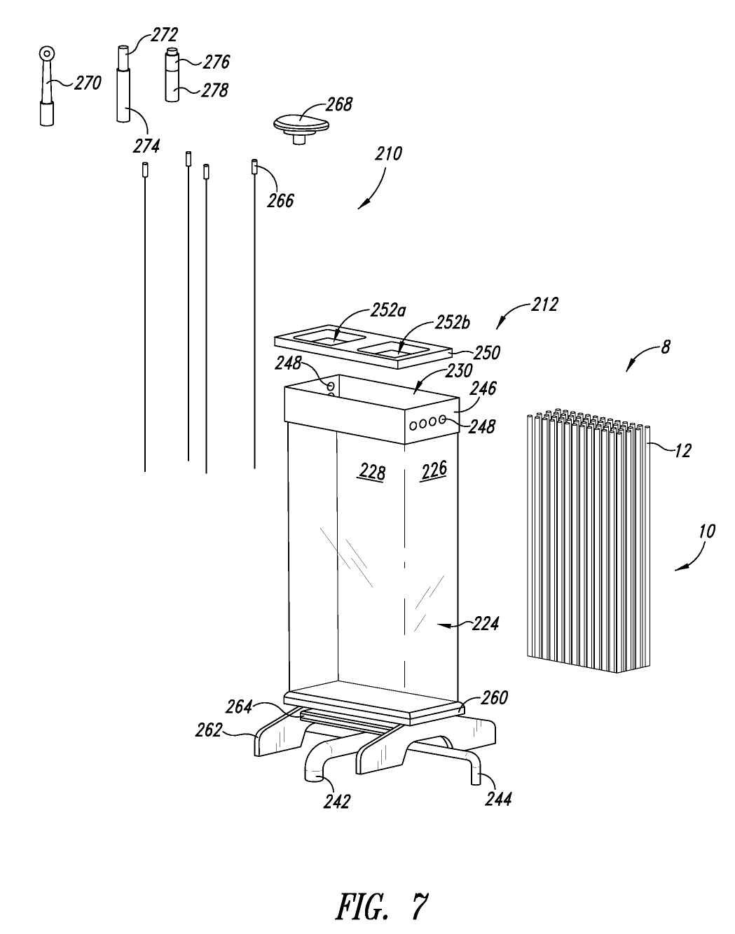

As shown in FIG. 6-8, a bioreactor system 210 can included one or more illumination assemblies 10. The bioreactor system 210 can also include a cover 250 including access ports 252a, 252b. The bioreactor system 210 may further include a housing structure 246, and one or more support structures 260, 262, 264.

In some embodiments, the bioreactor system 210 may include at least one container 224 having an exterior surface 226 and an interior surface 228. In some embodiments, the interior surface 228 defines an isolated space 230 adapted to retain biomasses, photosynthetic organisms, living cells, biological active substances, and the like. For example, the isolated space 230 defined by the interior surface 228 of the container 224 may be adapted to retain a plurality of photosynthetic organisms and cultivating media. The isolated space 230 can be adapted to, for example, serve as reservoir or a collection region for holding biomass-producing material.

The bioreactor 212 may take a variety of shapes, sizes, and structural configurations, as well as comprise a variety of materials. For example, the bioreactor 212 may take a cylindrical, tubular, rectangular, polyhedral, spherical, square, pyramidal shape, regular shape, irregular shape, and the like, or combinations thereof, as well as other symmetrical and asymmetrical shapes. In some embodiments, the bioreactor 212 may comprise at least a cross-section of substantially any shape including but not limited to circular, triangular, square, rectangular, polygonal, regular shape, irregular shape, and the like, as well as other symmetrical and asymmetrical shapes. In some embodiments, the bioreactor 212 may take the form of an enclosed vessel having one or more enclosures and/or compartments capable of sustaining and/or carrying out a chemical process such as, for example, the cultivation of photosynthetic organisms, organic matter, biochemically active substances, and the like.

Example of the materials useful for making the container 224 of the bioreactor 212 include but are not limited to, translucent materials, transparent materials, optically conductive materials, glass, plastics, polymer materials, and the like, or combinations or composites thereof, as well as other materials such as stainless steel, Kevlar, and the like, or combinations or composites thereof. Further example of suitable materials include concrete, (including, for example, concrete blocks, prestressed concrete, precasted concrete, pre-formed concrete, and the like), fiberglass, vinyl, polyvinyl chloride (PVC) plastic, metal, polyurethane foam, and the like, or other suitable building materials.

In some embodiments, the container 224 may comprise one or more transparent, translucent, or light-transmitting materials adapted to allow light to pass from the exterior surface to a plurality of photosynthetic organisms and cultivation media retained in the isolated space 230. In some further embodiments, a substantial portion of the container 224 comprises at least one of a transparent, translucent, or light-transmitting material. Examples of transparent, translucent, or light-transmitting materials include but are not limited to glasses, PYREX® glasses, plexiglasses, acrylics, polymethacrylates, plastics, polymers, and the like, or combinations or composites thereof.

The bioreactor system 210 may also include any suitable illumination systems 8, including one or more illumination assemblies 10 such as, for example, those described herein. In some embodiments, the illumination systems 8 comprises one or more optical waveguides 12 for providing light energy to at least some of a plurality of photosynthetic organisms retained in the isolated space 230.

In some embodiments, at least some of the one or more optical waveguides 12 include a plurality of structures 26 located proximate the first end 16 of the waveguides 12. In some embodiments, the plurality of structures 26 are configured to direct the first amount of light energy from the solar energy collector system 104 and the second amount of light energy from the plurality of light sources 14 along the interior 20 of the at least one substantially optically transparent cylindrical waveguide 12. In some embodiments, a plurality of light-diffusing structures 28 are located along the outer surface 22 of at least some of the optical waveguides 12. The plurality of light-diffusing structures are configured to guide at least a portion of the first and the second amounts of light directed along the interior of, for example, at least one substantially optically transparent cylindrical waveguide 12 to the exterior of the at least one substantially optically transparent cylindrical waveguide 12, and to supply the first amount of light and the second amount of light to at least some of a plurality of photosynthetic organisms retained in the isolated space 230.

In some embodiments, the one or more illumination assemblies 10 may be optically coupled to a solar collector system 104 for collecting sunlight and directing the light into the illumination system 8. In one embodiment, the solar collector system 104 is optically coupled via a fiber optic cable system 108 that is capable of receiving and routing sunlight into the one or more optical waveguides 12 as described in, for example, U.S. Pat. No. 5,581,447.

In some embodiments, the illumination assemblies 10 are adapted to supply light energy to at least some of a plurality of photosynthetic organisms retained in the isolated space 230. In some embodiments, the illumination assemblies 10 are configured to provide at least a first and a second light-emitting pattern. For example, in some embodiments, the illumination assemblies 10 can cycle through ON and OFF periods. In some embodiments, the illumination assemblies 10 can provide light energy to a first region of the bioreactor for a first period of time, and provide light energy to a second region of the bioreactor for a second period of time. The illumination assemblies 10 may further operate to produce at least a first illumination intensity level and a second illumination intensity level different from the first. In some embodiments, the second amount of light has at least one characteristic (e.g., light intensity, illumination intensity, light-emitting pattern, peak emission wavelength, on-pulse duration, and/or pulse frequency) different from a like characteristic of the first amount of light. In some other embodiments, the second amount of light has the same characteristics as the first amount of light.

In some embodiments, the bioreactor system 210 may include one or more mirrored and/or reflective surfaces received in and/or formed on the interior 230 of the bioreactor 212. In some embodiments, a portion of the interior surface 228 of the bioreactor 212 may include mirrored and/or reflective surfaces such as, for example, a film, a coating, an optically active coating, a mirrored and/or reflective substrate, and the like. In some embodiments, the bioreactor 212 may include housing structures including one or more mirrored and/or reflective surfaces in a portion adjacent to the exterior surface 226 of the container 224.

In some embodiments, the one or more mirrored and/or reflective surfaces may be configured to maximize distribution of light emitted by the illumination assemblies 10.

The illumination assemblies 10 may comprise a single optical waveguide 12, or may comprise multiple optical waveguides 12. The illumination assemblies 10 may come in a variety of shapes and sizes. In some embodiments, the illumination assemblies 10 may comprise a cross-section of substantially any shape including circular, triangular, square, rectangular, polygonal, regular or irregular shapes, and the like, as well as other symmetrical and asymmetrical shapes. In some embodiments, the cylindrical optical waveguides 12 may be optically coupled to each other via one or more optical fibers.

In some embodiments, the illumination system 8 is operable to provide a photon flux suitable for cultivating at least one of biomasses, photosynthetic organisms, living cells, biological active substances, or the like. In some embodiments, the illumination system 8 is operable to provide a photon flux of about 100 micromoles per square meter per second to about 1400 micromoles per square meter per second. In some embodiments, the illumination system 8 is operable to provide a photon flux of about 200 micromoles per square meter per second to about 600 micromoles per square meter per second. In some embodiments, optimal photosynthetic efficiency is achieved with a photon flux in the range of about 200 micromoles per square meter per second to about 400 micromoles per square meter per second. In some embodiments, a photon flux above 1400 micromoles per square meter per second may result in an inhibition of photosynthesis.

Certain biomasses, for example, plants, algae, and the like comprise two types of chlorophyll, chlorophyll a and chlorophyll b. Each type typically possesses a characteristic absorption spectrum. In some cases the spectrum of photosynthesis of certain biomasses is associated with (but not identical to) the absorption spectra of, for example, chlorophyll. For example, the absorption spectra of chlorophyll a may include absorption maxima at about 430 nm and 662 nm, and the absorption spectra of Chlorophyll b may include absorption maxima at about 453 nm and 642 nm. In some embodiments, the one or more illumination assemblies 10 may be configured to provide one or more peak emissions associated with the absorption spectra of chlorophyll a and chlorophyll b.

In some embodiments, the one or more illumination assemblies 10 include a plurality of optical waveguides 12 to optically couple a source of light located in the exterior of the bioreactor 212 to a portion of the illumination system 8 received in the isolated space 230. In some embodiments, the optical waveguides 12 take the form of a plurality of optical fibers.

In some embodiments, the illumination system 8 may further include at least one optical waveguide 12 on the exterior surface 226 of the container 224 optically coupled to the illumination system 8. The at least one optical waveguide 12 may be configured to optically couple a source of solar energy to at least a portion of the illumination system 8 received in the isolated space 230. The source of solar energy may include a solar collector system 104 including a solar collector and a solar concentrator assembly 109 (shown in dashed line) optically coupled to the solar collector and the portion of the illumination system 8. The solar concentrator assembly can be configured to concentrate solar energy provided by the solar collector and, for example, to provide the concentrated solar energy to a portion of the illumination system 8 received in the isolated space 230. The solar concentrator assembly 109 can include one or more lenses (e.g., Fresnel lenses, converging lenses, biconvex lenses, and the like), mirrors, and optical trains (e.g., an array of optical elements such as lenses), as well as other optical elements and solar concentrators.

Any suitable solar collector or solar concentrator may be used with any of the disclosed systems, devices, and methods. Further examples of solar collectors or solar concentrators include, but are not limited to, solar troughs (e.g., parabolic troth concentrators, and the like), solar dishes (e.g., parabolic reflectors, parabolic dishes, and the like), flat-plate solar collectors, stationary or mobile concentrating collectors, solar power towers, and the like.

In some embodiments, the one or more illumination assemblies 10 are encapsulated in a medium having a first index (n₁) of refraction and the growth medium has a second index of refraction (n₂) such that the differences between n₁ and n₂, at a given wavelength selected from a spectrum ranging from about 440 nm to about 660 nm, is less than about 1. Examples of the medium having a first index (n₁) of refraction include mineral oil. Mineral oil may also serve to cool the LEDs and prevent water migration into the electronics, for instance in the event of a panel case seal failure.

In some embodiments, the bioreactor 212 may further include conductivity probe 270. The bioreactor system 210 may further include one or more sensors including dissolved oxygen sensors 272, 274, pH sensors 276, 278, a level sensor 268, CO₂ sensors, oxygen sensors, and the like. The bioreactor system 210 may also include one or more thermocouples 266. The bioreactor 212 may include, for example, inlet and/or outlet ports 248, and inlet and/or outlet conduits 240, 242, 244, for providing or discharging process elements, nutrients, gasses, biomaterials, and the like, to and from the bioreactor 212.

In some embodiments, the bioreactor system 210 can be coupled to a source of growth media, can be adapted to receive growth media within the isolated space 230 of the container 224, or may include growth media received in the isolated space 230 of the container 224, or any combinations thereof. Growth media may be for freshwater, estuarine, brackish or marine bacterial or algal species and/or other microorganisms or plankton. The growth media may include salts, such as sodium chloride and/or magnesium sulfate, macronutrients such as nitrogen and phosphorus containing compounds, micronutrients such as trace metals, for example, iron and molybdenum containing compounds and/or vitamins, such as Vitamin B₁₂. The growth media may be modified or altered to accommodate various species and/or to optimize various characteristics of the cultured species, such as growth rate, protein production, lipid production and carbohydrate production.

In some embodiments, the bioreactor system 210 can include a second illumination system adjacent to the exterior surface 226 of the container. The second illumination system may comprise at least one light-emitting substrate configured to provide light to at least some of the plurality of photosynthetic organisms retained in the isolated space 230 and located proximate a portion of the interior surface 226 of the container 224. In some embodiments, the second illumination system includes at least one light-emitting substrate located on a housing structure configured to enclose the bioreactor 212.

As shown in FIG. 8, the bioreactor system 210 may further include a control system 300 operable to control the voltage, current, and/or power delivered to the bioreactor 212, as well as automatically control at least one process variable and/or a stress variable that alters or affects the growth and/or development of an organism (e.g., changing stress variable to induce nutrient deprivation, nitrogen-deficiency, silicon-deficiency, pH, CO₂ levels, oxygen levels, degree of sparging, or other conditions that affect growth and/or development of an organism). In some embodiments, stressing the photosynthetic organism affects, for example, a lipid content. Examples of stressing include changing stress variable to induce nutrient deprivation, nitrogen-deficiency, silicon-deficiency, pH, CO₂ levels, oxygen levels, degree of sparging, or other conditions that affect growth and/or development of an organism, and the like. See, e.g., Spoehr & Milner: 1949, Plant Physiology 24, 120-149. In particular, nitrogen deficiency reduced growth rates and resulted in high oil content: 1 Tornabene et al: 1983, Enzyme and Microbial Technology, 435-440; 2—Lewin: 1985, Production of hydrocarbons by micro-algae: isolation and characterization of new and potentially useful algal stains, SER1/CP-231-2700, 43-51; 3—Zhekisheva et al: 2002, Journal of Phycology, 325-331. Silicon deficiency in diatoms yielded similar results: Tadros & Johansen: 1988, Journal of Phycology, 445-452. In some embodiments, the method further includes temperature stressing the photosynthetic organism. In some embodiments, the bioreactor 212 may operate under strict environmental conditions that require controlling one or more process variables associated with cultivating and/or growing a photosynthetic biomass. For example, the bioreactor system 210 may include one or more sub-systems for controlling gas flow rates (e.g., air, oxygen, CO₂, and the like), effluent streams, temperatures, pH balances, nutrient supplies, other organism stresses, and the like.

The control system 300 may include one or more controllers 302, for example, microprocessors, digital signal processor (DSPs) (not shown), application-specific integrated circuits (ASICs) (not shown), field programmable gate arrays (FPGAs) (not shown), and the like. The control system 300 may also include one or more memories, for example random access memory (RAM) 304, read-only memory (ROM) 306, and the like, coupled to the controllers 302 by one or more busses. The control system 300 can include a wide range of stored programs based on the desired production cycle. The control system 300 may further include one or more input devices 308 (e.g., a keypad, touch-screen display, and the like). The control system 300 may also include discrete and/or an integrated circuit elements 310 to control the voltage, current, and/or power. In some embodiments, the control system 300 is configured to control at least one of light intensity, illumination intensity, a light-emitting pattern, a peak emission wavelength, an ON-pulse duration, and a pulse frequency associated with one or more illumination assemblies 10 based on a measured optical density.

The control system 300 can be a closed loop or open loop system. For example, the closed loop control system 300 can control operation based upon feedback signals from one or more sensors configured to detect light intensity, the presence and/or amount of light energy, temperature (e.g., temperature of the biomass), and combinations thereof as well as other measurable parameters of interest. The sensors can transmit one or more signals indicative of the measured parameter(s) of interest. Based on those signals, the control system 300 can adjust the production cycle.

Alternatively, the control system 300 can be an open loop system wherein the operation of the bioreactor is set by user input. For example, the amount of light energy delivered to the biomass may be set to a fixed power mode by utilizing the control system 300. In some embodiments, the control system 300 can include a program that ensures that proper biomass production is sustained throughout the entire day, including the nighttime hours. It is contemplated that the control system 300 can be switched between a closed loop system and an open loop system.

The bioreactor system 210 may further include a variety of controller systems 314, sensors 312, as well as mechanical agitators 314, and/or filtration systems, and the like. These devices may be controlled and operated by the central control system 300. In some embodiments, the one or more sensors 312 may be operable and/or configured to determine at least one of a temperature, pressure, light intensity, optical density, opacity, gas content, pH, fluid level, sparging gas flow rate, salinity, fluorescence, absorption, mixing, and/or turbulence. The controller 300 and/or 314 may be configured to control at least one of an illumination intensity, illumination pattern, peak emission wavelength, ON-pulse duration, and/or pulse frequency based on a sensed temperature, pressure, light intensity, optical density, opacity, gas content, pH, fluid level, sparging gas flow rate, salinity, fluorescence, absorption, mixing, and/or turbulence.

The bioreactor system 210 may also include sub-systems and/or devices that cooperate to monitor and possibly control operational aspects such as the temperature, salinity, pH, CO₂ levels, O₂ levels, nutrient levels, and/or a light supply, and the like. In some embodiments, the bioreactor system 210 may include the ability to increase or decrease each aspect or parameter individually or in any combination, for example, temperature may be raised or lowered, gas (e.g., CO₂, O₂, etc.) levels may be raised or lowered, pH, nutrient levels, and light, may be raised or lowered. The light can be natural or artificial. Some general lighting control aspects include controlling the duration that the light operates on portions of, for example, an algal mass in the bioreactor 212, cycling the light (to include periods of light and dark), for example, artificial light, to extend the growth of the algae past daylight hours, controlling the wavelength of the light, controlling the lighting patterns, and/or controlling the intensity of the light. Lighting control may also include controlling one or more filters, operatives, masks, shades, and/or levers, particularly where the light is natural.

The bioreactor system 210 may further include a carbon dioxide recovery system 316 for recovering, treating, extracting, utilizing, scrubbing, cleaning, and/or purifying a carbon dioxide supply from, for example, flue gas of an industrial source (e.g., an industrial plant, an oil field, a coal mine, and the like).

The bioreactor system 210 may further include one or more nutrients supply systems 318, solar energy supply systems 320, and heat exchange systems 322. Examples of nutrients supply systems 318 include, but are not limited to, wastewater, storm water run-off, as well as water from lakes, ponds, or streams, and the like.

Biomasses such as, for example, algal biomasses many beneficially ameliorate the effects of pollutants or act as pollution control agents to treat wastewater, storm water run-off, lakes, ponds, or streams, and the like. For example, algal biomasses may help remove, capture, or treat pollutants (e.g., fertilizers) carried in the nutrients supply systems 318. Once treated, the water may be subsequently returned to the lakes, ponds, or streams.

The nutrients supply systems 318 may include, or be part of, one or more effluent and/or nutrient streams. An effluent is generally regarded as something that flows out or forth, like a stream flowing out of a body of water. For example, this includes, but is not limited to, discharged wastewater from a waste treatment facility, brine wastewater from desalting operations, and the like. In the context of algae cultivation, an effluent stream may contain nutrients to feed algae present inside and/or outside of a bioreactor 212. In one embodiment, the effluent stream includes biological waste or waste sludge from a waste treatment facility (e.g., sewage, landfill, animal, slaughterhouse, toilet, outhouse, portable toilet waste, and the like). Such an effluent stream (including the CO₂ produced by the bacteria within such waste) can be directed to the algae, where the algae remove nitrogen, phosphate, and carbon dioxide (CO₂) from the stream. In another embodiment, the effluent stream comprises flue gases from power plants. The algae remove the CO₂ and various nitrogen compounds (NOₓ) from the flue gases. In each of the foregoing embodiments, the algae use the CO₂, in particular, for the process of photosynthesis. The oxygen produced by the algae during the photosynthetic process could be utilized to, for example, promote further bacterial growth and CO₂ production in a waste effluent stream. Furthermore, it is understood that the effluent streams can be seeded with a variety of additional nutrients and/or biological material to stimulate and enhance the growth rate, photosynthetic process, and overall cultivation of the algae.

The solar energy supply systems 320 may collect and/or supply sunlight, as well as direct light into the bioreactor 212. In some embodiments, solar energy supply systems 320 include a solar energy collector system 104 including a solar energy collector and a solar energy concentrator including a plurality of optical elements configured and positioned to collect and concentrate sun light.

In some embodiments, the solar energy supply systems 320 may be further used to generate power. For example, excess solar energy may be use to generate power. Solar light energy may be converted into electrical energy using, for example, solar (photovoltaic) cells. In some embodiments, the solar energy may be use to heat fluids and produce steam. The steam, in turn, may be converted to mechanical energy in a turbine, and into electricity using, for example, a conventional generator coupled to the turbine.

In one embodiment of a bioreactor 212 utilizing solar energy directed into fiber optics, only photosynthetically active radiation (PAR) light is passed on to the growing algae. The UV (ultraviolet) and IR (infrared) wavelengths are filtered out. In other embodiments, UV-IR wavelengths are use to generate power using for example solar (photovoltaic) cells or use to heat fluids and produce steam that is consequently use to generate electricity using, for example, a conventional generator coupled to a turbine.

The heat exchange system 322 typically controls and/or maintains a constant temperature within the bioreactor 212. For example, temperature within the bioreactor may be lowered to stress the algae to promote oil production, etc., at the end of a growth cycle. In some embodiments, the heat exchange system 322 and the control system 300 operate to maintain a constant temperature in the bioreactor 212 to sustain a bioprocess within.

The bioreactor system 210 may further include a biomass and/or oil recovery system 324, and a biofuel production system 326.

The biomass and/or oil recovery system 324 may take the form of an algae oil recovery system and may further include an extraction system, such as a press device or a centrifuge device to extract, for example, lipid, a medical compound, and/or a labeled compound from photoorganisms (e.g., algae, and the like). Various methods and techniques may be used for causing photoorganisms to produce medical compounds and/or labeled compounds (e.g., isotopically labeled compounds, and the like).

The extraction system may be located within or outside of the bioreactor 212. Additionally or alternatively, the extraction system may comprise an extractant selected from chemical solvents, supercritical gases or liquids, hexane, acetone, liquid petroleum products, and primary alcohols. In other embodiments, the extraction system includes a means for genetically, chemically, enzymatically or biologically extracting, or facilitating the extraction of, lipid from the algae.

In some embodiments, a conversion system may be operably coupled to the extraction system to receive the lipid and convert the lipid to biofuel. In one embodiment, the conversion system includes a transesterification catalyst and an alcohol. In other embodiments, the conversion system includes an alternate means for genetically, chemically, enzymatically, or biologically converting the lipid to biofuel. In some embodiments, various enzymes may be utilized to break down the algal cell structure prior to extraction, thereby facilitating the subsequent extraction acts, e.g., minimizing the energy required in a physical extraction process such as a pressing or centrifuging.

The biofuel production system 326 may include various technologies for processing and/or refining biofuel from biomasses. For example, a catalytic cracking process can be used to produce other desirable fuel products and/or by-products. Catalytic cracking breaks the complex hydrocarbons in the biofuel into simpler molecules to create a higher quality and greater quantity of a lighter, more desirable fuel product, while also decreasing an amount of residuals in the biofuel. The catalytic cracking process rearranges the molecular structure of hydrocarbon compounds in the biofuel to convert heavy hydrocarbon feedstock into lighter fractions such as kerosene, gasoline, LPG, heating oil, and petrochemical feedstock.

In some embodiments, catalytic cracking process may be advantageous over thermal cracking processes because the yield of improved-quality fuels can be achieved under much less severe operating conditions than in thermal cracking, for example. The three types of catalytic cracking processes are fluid catalytic cracking (FCC), moving-bed catalytic cracking, and Thermofor catalytic cracking (TCC). The catalytic cracking process is very flexible, and operating parameters can be adjusted to meet changing product demand. In addition to cracking, catalytic activities include dehydrogenation, hydrogenation, and isomerization as described in, for example, U.S. Pat. No. 5,637,207.

Biodiesels and the production of biodiesels from, for example, algae can be used in a variety of applications. Such applications include the production of biodiesel and subsequent refinement to other fuels, including those that could be used as, or as a component of, jet fuels (e.g., kerosene). Such production could occur using catalytic cracking or any other known process for generating such fuels from the biofuels produced by algae. In one embodiment, such refining occurs as part of the same system used to extract the biofuels from the algae. In another embodiment, the biofuels are transported by truck, train, pipe, or other means to a second location where refining of the biofuel into other fuels such as those noted above occurs.

In some embodiments, the bioreactor system 210 takes the form of a bio-system adapted to produce biofuel from algae. The bio-system includes a bioreactor 212 with an illumination system 8 that is arranged to direct an amount of light on at least some algae located within the bioreactor 212. The algae can be brought into the bioreactor 212 via an effluent stream or the algae may be present within the bioreactor 212 prior to effluent introduction or may be seeded prior to effluent or nutrient stream introduction, concurrently therewith or subsequently. At least one or more filters can be positioned in the bioreactor 212 to filter non-algae type particulates from the effluent stream and/or separate the algae based on some characteristic or physical property of the algae.

The illumination system 8 may be configured within the bioreactor 212 to increase the photosynthetic rate of the algae, and thus increase the yield of lipids from the algae. The bio-system may further include the control system 300 coupled to and/or located within the bioreactor 212 to monitor and/or control at least one environmental condition within the bioreactor 212, for example, the temperature, humidity, effluent stream flow rate, and the like. In some embodiments, the control system 300 controls one or more sensors 312 (e.g., temperature sensor) located within a first region of the bioreactor 212. In some embodiments, an optical density or opacity measurement device measures the specific gravity and/or concentration of at least some of the algae just before it enters, or just after it enters, the bioreactor 212.

In some embodiments, a light source is optically coupled to at least a portion of the illumination system 8. In one embodiment, the light source comprises a plurality of LEDs that provide artificial light to at least some of the algae. In another embodiment, the light source is a solar collector system 104 that collects sunlight. The solar collector is optically coupled to the illumination system 8, which comprises a network of fiber optic waveguides and optical switches to route, guide, and eventually direct at least a portion of the light collected by the solar collector toward at least some of the algae within the bioreactor 212.

In yet additional embodiments, the bioreactor system 210 comprises one or more light sources that can alternate between artificial and natural light. In such an embodiment, the system can be configured to utilize natural light during periods of solar light availability and automatically or manually switch to artificial light when insolation or solar output falls below a target level. Further, one, two, or more light sources could perform both natural and artificial lighting or a first light source could provide the artificial light source, while a second light source could provide the natural light. Alternatively, the light source or sources may concurrently operate at various levels to maximize light availability to an organism (e.g., algae).

In some embodiments, an agitation system is arranged in the bioreactor system 210 to agitate, circulate, or otherwise manipulate the water, algae, effluent nutrient stream, flue gases, or some combination thereof. The agitation system can be configured so that the algae is continually mixed, where at least some of the algae is exposed to light while other algae is not exposed to light (e.g., the other algae is placed into a dark cycle). The agitation system may operate to advantageously reduce an amount of light-providing surface area to a volume of the algae within the bioreactor 212, yet still obtain a desired amount of lipid production. Additionally or alternatively, light/dark cycling may be accomplished by turning the light source ON/OFF).

In various applications, a bioreactor system 210 comprising both a bioreactor 212 and an extraction system 324, and optionally a system for refining or processing biofuel 326, may be attached to a waste treatment facility such that the bioreactor system 210 utilizes an effluent stream from the waste treatment facility as a nutrient source for the algae. In some embodiments, the algae is subsequently harvested for biofuel that may be utilized to power the waste treatment facility.

In other applications, a bioreactor system 210 comprising both a bioreactor 212 and an extraction system 324, and optionally a system for refining or processing biofuel 326, may be incorporated into an automobile, train, airplane, ship, or any other vehicle having an internal combustion engine. In such applications, the CO₂ produced by the engine may be utilized by, for example, a recovery system 316 as a nutrient source for the algae, and the heat generated by the engine may be utilized to promote algal growth, for example, by incorporating thermoelectric devices to convert the heat into electricity to power the bioreactor light source, and/or maintaining a desired temperature profile.

In other embodiments, a bioreactor system 210 comprising both a bioreactor 212 and an extraction system 324, and optionally a system for refining or processing biofuel 326, may be utilized in concert with a power plant. In such embodiments, the excess heat generated at the power plant may be utilized to heat and dry the harvested algae. In certain embodiments, particularly in embodiments wherein the harvested algae has a hydrocarbon content greater than about 70%, the harvested algae may be directly utilized as fuel in the power plant without the need for any extraction, refining, or processing.

In other embodiments, a bioreactor system 210 in the form of a portable bio-system comprising both a bioreactor 212 and an extraction system 324, and optionally a system for refining or processing biofuel 326, may be shipped to, dropped into, or delivered to a remote location or disaster zone as away of providing fuel for emergency use.

Although growing and harvesting algae (broadly referred to as biomass) for biofuel or biodiesel, feedstock, and/or other purposes has been generally known since at least the late 1960s, there has been a renewed interest in this technology in part because of rising petroleum costs. Microscopic algae (hereinafter referred to as micro-algae) are regarded as being superb photosynthesizers and many species are fast growing and rich in lipids, especially oils. Some species of micro-algae are so rich in oil that the oil accounts for over fifty percent of the micro-algae's mass. These and other interesting qualities and characteristics of micro-algae are discussed in, for example, “An Algae-Based Fuel” by Olivier Danielo, Biofutur, No. 255 (May 2005).

Two types of micro-algae that are generally known to produce a high percentage of oil are Botryococcus braunii (commonly abbreviated to “Bp”) and Diatoms. Diatoms are unicellular algae generally placed in the family Bacillariophyceae and are typically brownish to golden in color. The cell walls of Diatoms are made of silica.

There are approximately 100,000 known species of algae around the world and it is estimated that more than 400 new species are discovered each year. Algae are differentiated mainly by their cellular structure, composition of pigment, nature of the food reserve, and the presence, quantity, and structure of flagella. Algae phyla (divisions) include, for example, blue/green algae (Cyanophyta), euglenids (Euglenophyta), yellow/green and golden/brown algae (Chrysophyta), dinoflagellates and similar types (Pyrrophyta), red algae (Rhodophyta), green algae (Chlorophyta), and brown algae (Phaeophyta).

In the production of biofuel, micro-algae is faster growing and can synthesize up to thirty times more oil than other terrestrial plants used for the production of biofuel, such as rapeseed, soybean, oil palm, wheat, or corn. One of the main factors for determining the yield or productivity of biofuel from micro-algae is the amount of algae that is exposed to sunlight.

Many types of algae produce by-products such as colorants, poly-unsaturated fatty acids, and bio-reactive compounds. These and other by-products of algae may be useful in food products, pharmaceuticals, supplements, and herbs, as well as personal hygiene products. In one embodiment, the algal by-product left over after lipid extraction is used to produce animal feed.

In some embodiments of the various embodiments of the systems, devices, and methods described herein, the algae utilized may be genetically modified to, for example, increase the oil content of the algae, increase the growth rate of the algae, change one or more growth requirements (such as light, temperature and nutritional requirements) of the algae, enhance the CO₂ absorption rate of the algae, enhance the ability of the algae to remove pollutants (e.g., nitrogen and phosphate compounds) from a waste effluent stream, increase the production of hydrogen by the algae, and/or facilitate the extraction of oil from the algae. See, e.g., U.S. Pat. Nos. 5,559,220; 5,661,017; 5,365,018; 5,585,544; 6,027,900; as well as U.S. Patent Application Publication No. 2005/241017.

As previously disclosed, the bioreactor system 210 may further include a control system 300 operable to control the voltage, current, and/or power delivered to the bioreactor 212, as well as automatically control at least one process variable and/or a stress variable that alters or affects the growth and/or development of an organism. For example, in some embodiments, the control system 300 is configured to control at least one of a light intensity, illumination intensity, light-emitting pattern, peak emission wavelength, on-pulse duration, and/or pulse frequency associated with the illumination assemblies 10 based on a measured optical density.

In some embodiments, the one or more illumination assemblies 10 are configured to supply an effective amount of light to a substantial portion of the plurality of photosynthetic organisms retained in the isolated space 230. In some embodiments, an effective amount of light comprises an amount sufficient to sustain a biomass concentration having an optical density (OD) value greater than from about 0.1 grams/liter to about 15 grams/liter. Optical density may be determined by having an LED on the surface of one panel and an optical sensor directly opposite on the surface of another panel.

In some embodiments, the illumination assemblies 10 are operable to provide a photon flux of about 100 micromoles per square meter per second to about 1400 micromoles per square meter per second. In some embodiments, the illumination assemblies 10 are operable to provide a photon flux of about 200 micromoles per square meter per second to about 600 micromoles per square meter per second. In some embodiments, optimal photosynthetic efficiency is achieved with a photon flux in the range of about 200 micromoles per square meter per second to about 400 micromoles per square meter per second. In some embodiments, a photon flux above 1400 micromoles per square meter per second may result in an inhibition of photosynthesis.

Alternatively, the initial sensor may be a separate device inside the medium. For each algae species, samples of the growth are taken and a concentration level is determined by filtering the algae and weighing the results. Samples are taken at a minimum of three different concentration levels and those values are corresponded to the optical readings from between the panels or device inside the medium and an algorithm is created using the data. Optical density can then be monitored optically and manipulated with the control system 300.

In some embodiments, an effective amount of light comprises an amount sufficient to sustain a photosynthetic organism density greater than 1 gram of photosynthetic organism per liter of cultivation media. In some embodiments, an effective amount of light comprises an amount sufficient to sustain a photosynthetic organism density greater than 5 grams of photosynthetic organism per liter of cultivation media. In some further embodiments, an effective amount of light comprises an amount sufficient to sustain a photosynthetic organism density ranging from about 1 gram of photosynthetic organisms per liter of cultivation media to about 15 grams of photosynthetic organisms per liter of cultivation media. In yet some other embodiments, an effective amount of light comprises an amount sufficient to sustain a photosynthetic organism density ranging from about 10 grams of photosynthetic organisms per liter of cultivation media to about 12 grams of photosynthetic organisms per liter of cultivation media.

The control system 300 may further be configured to automatically control at least one process variable. For example, the control system 300 can be configured to automatically control at least one of a bioreactor interior temperature, bioreactor pressure, pH level, nutrient flow, cultivation media flow, gas flow, carbon dioxide gas flow, oxygen gas flow, light supply, or the like.

In some embodiments, the bioreactor 212 comprises one or more effluent streams providing fluidic communication of gasses, liquids, and the like between the exterior and/or interior of the bioreactor 212. In some embodiments, the bioreactor 212 comprises an enclosed system wherein no effluent streams go in or out on a continual basis.

Bioreactor systems 212 often operate under strict environmental conditions. Thus, there are many components, assemblies, and/or sub-systems that comprise the bioreactor system 210, for example, sub-systems for controlling gasses (e.g., air, oxygen, CO₂, etc.) in and out of the bioreactor, effluent streams, flow rates, temperatures, pH balances, etc. Bioreactor systems 10 may employ a variety of sensors, controllers, mechanical agitators, and/or filtration systems, etc. These devices may be controlled and operated by a central control system. It is understood that the design and configuration of a bioreactor system 210 can be complex and varied depending on the location and/or purpose of the bioreactor 212.

In one embodiment, the bioreactor system 210 includes sub-systems and/or devices that cooperate to monitor and possibly control operational aspects such as the temperature, salinity, pH, CO₂ levels, O₂ levels, nutrient levels, and/or the light. In further aspects, the bioreactor system 210 may include the ability to increase or decrease each aspect or parameter individually or in any combination, for example, temperature may be raised or lowered, gas levels may be raised or lowered (e.g., CO₂, O₂, etc.), pH, nutrient levels, light, etc., may be raised or lowered. The light can be natural or artificial. Some general lighting control aspects include controlling the duration that the light operates on portions of the algae in the bioreactor 212, cycling the light (to include periods of light and dark), for example, artificial light, to extend the growth of the algae past daylight hours, controlling the wavelength of the light, and/or controlling the intensity of the light. These aspects, among others, are described in further detail below.

In some embodiments, the bioreactor 212 is operable for processing micro-algae. The bioreactor 212 may include a number of levels, channels, or tubes. In various embodiments, the levels may comprise stackable algae panels. A first surface layer of micro-algae is photosynthesized on a first level, a second surface layer of micro-algae is photosynthesized on a second level, and so on. In some embodiments, the bioreactor 212 may have “1-n” levels, where n is greater than 2.

In one embodiment, a source directs a stream of micro-algae to the bioreactor where the micro-algae are directed to the different levels or channels. The micro-algae may be separated based on a number of criteria, such as the specific density, size, and/or type of micro-algae. In addition, flue gasses rich in CO₂ may be directed into the bioreactor to enrich the micro-algae and provide the necessary amount of CO₂ for the photosynthetic process to occur, as well as to assist in removing CO₂ and other gases from the flue gas.

In another embodiment, the algae is seeded or pre-placed in the bioreactor 212. An effluent stream is directed into the bioreactor 212 to provide nutrients to the algae. The effluent stream can be a stream of wastewater as described above. Additionally or alternatively, flue gasses rich in CO₂ may be directed into the bioreactor 212 to enrich the micro-algae and provide the necessary amount of CO₂ for the photosynthetic process to occur.

The levels or channels of the bioreactor 212, in which the algae is cultivated, can have a variety of configurations and/or cross-sectional shapes. For example, a first level or channel may be narrow in places and wide in other places to control an amount of light penetration on the algae. For example, narrow levels or channels can be arranged to provide a dark cycle for the algae, whereas the wide levels or channels permit the algae to cover a larger surface area so that more of the algae is exposed to the light.

The photosynthetic process can employ both dark and light cycles. Dark cycles allow the algae to process a photon of light. During the light cycle, the algae absorb photons of light. By way of example, once a photon of light is absorbed, which happens in a range of about 10⁻¹⁴ to 10⁻¹⁰ seconds, it takes approximately 10⁻⁶ seconds for the algae to perform photosynthesis and reset itself to be ready to absorb another photon. Accordingly, the levels or channels and/or illumination system can be arranged in the bioreactor 212 to advantageously control the light and dark cycles to increase the photosynthetic efficiency of the algae therein.

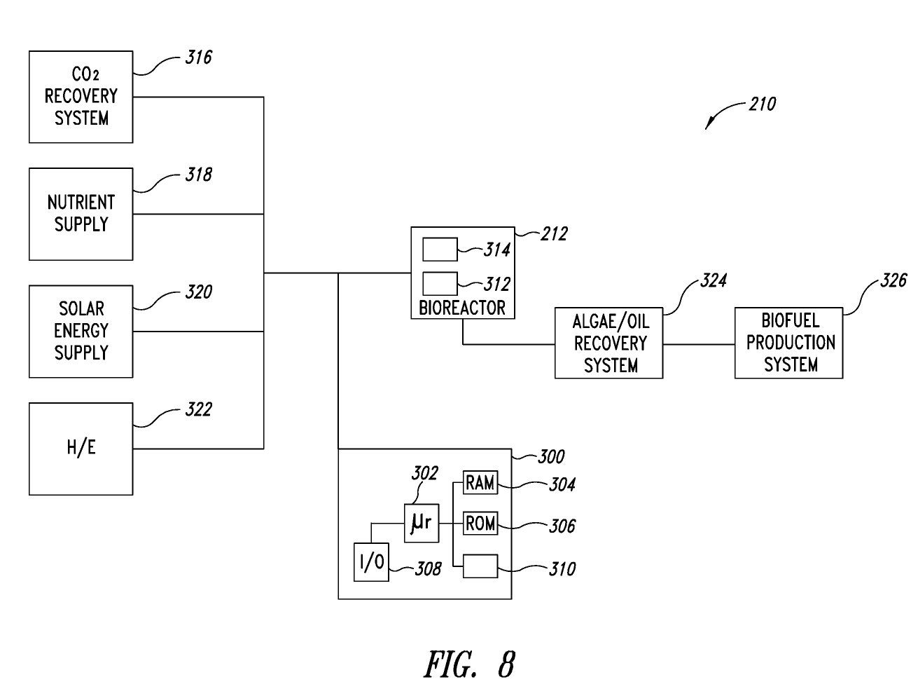

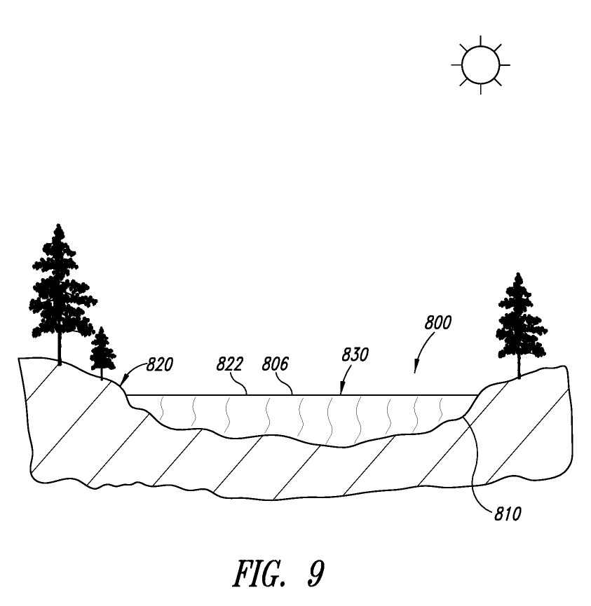

FIGS. 9-15 show open bioreactor systems 800, 840, 880, according to multiple embodiments, that can be similar to the closed bioreactor systems disclosed herein. Generally, open bioreactor systems 800, 840, 880 can be exposed to the surrounding environment. Natural resources (e.g., ambient gases such as air, ambient fluids such as rain water or runoff, sunlight, thermal energy, airflow, and the like) in the surrounding environment may be used to affect the production of biomass. The holding capacity, configuration (e.g., average depth of holding chamber or reservoir), and other processing parameters of the bioreactor systems 800, 840, 880 can be selected based on the desired biomass production rate and type of biomass producing material utilized. Accordingly, natural resources may be utilized to reduce manufacturing costs, improve the quality of the biomass, yield high production rates, and the like. Because a biomass producing material in open bioreactors can utilize natural resources, manufacturing costs of biomass passively produced in the open bioreactor may be less than manufacturing costs of biomass produced in closed bioreactors employing primarily actively delivered resources.

FIG. 9 shows an open-air bioreactor 800 filled with biomass producing material 806. The illustrated bioreactor 800 includes a reservoir 810 holding the biomass producing material 806 such that a sufficient amount of sunlight and a sufficient amount of ambient gases are exposed to the biomass producing material 806 to support a wide range of bioreactions (e.g., small to large scale bioreactions).

To reduce the manufacturing cost of the open bioreactor 800, the reservoir 810 can be a natural reservoir, such as a lake, pond, stream, canal, or other naturally occurring body of water. Various types of additives can be disposed into the water to produce a desired biomass producing material. In some embodiments, the water can be drained from the reservoir 810 and replaced with biomass producing material.

As shown in FIG. 9, the shore 820 surrounding the upper surface 830 of the biomass producing material 806 defines an “opening” or exposure window 822. The biomass producing material 806 (e.g., algae) can utilize sunlight passing through the opening 822, although light systems or other auxiliary systems can be added to the open bioreactor 800, if needed or desired. For example, in some embodiments, the open bioreactor 800 may include an illumination system 8 received in the reservoir 810. In some embodiments, the reservoir 810 is an artificial reservoir that can be formed at a location suitable for biomass production. Advantageously, an artificial reservoir 810 can be rapidly installed at a wide variety of locations for convenient biomass production near, for example, a consumption site. For example, for a facility (e.g., a manufacturing plant) that consumes a significant amount of biomass product, the open bioreactor 800 can be installed near the facility to minimize or limit biomass transportation costs.

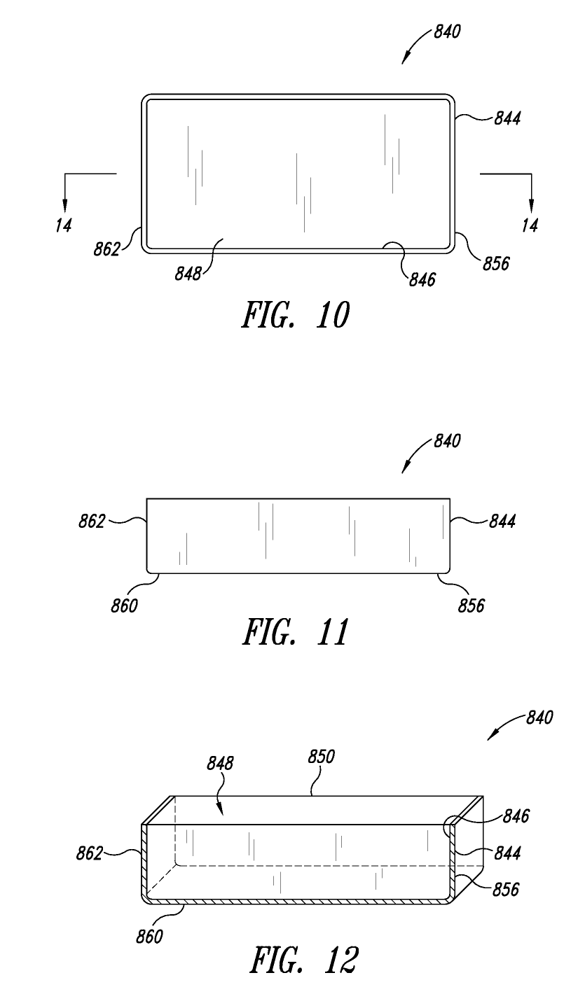

Referring to FIGS. 10-15, in some embodiments, the bioreactor 840 is a portable open-air tank having an exterior surface 844 and an interior surface 846. The interior surface 846 defines a reservoir or chamber 848 for holding the biomass producing material. The bioreactor 840 includes an opening 850 through which biomass producing material or its components can be delivered. A wall 856 of the bioreactor 840 can comprise transparent or translucent materials to allow additional ambient light to reach the biomass producing material. As used herein, the term “wall” is broadly construed to include, without limitation, a bottom, sidewall, and other structures suitable for forming a reservoir or holding chamber. The illustrated wall 856 includes a bottom 860 and a sidewall 862 extending away from the bottom 860.

The portable bioreactor 840 can be conveniently transported to a wide range of locations for on-site biomass production. The holding capacity of the chamber 848 can be selected based on biomass production rate. For example, the chamber 848 can hold a few gallons to thousands of gallons of biomass producing material. Additionally, the average depth, cross-sectional area (e.g., the cross-sectional area of the chamber 848 taken generally perpendicular to an upper surface of biomass producing material when the chamber 848 is filled), and other dimensions of the bioreactor 840 can be varied as desired.

An array of open and/or closed bioreactors can be used for a highly scalable biomass production system. The number and type of bioreactors can be periodically changed in order to efficiently make a desired amount of biomass product.

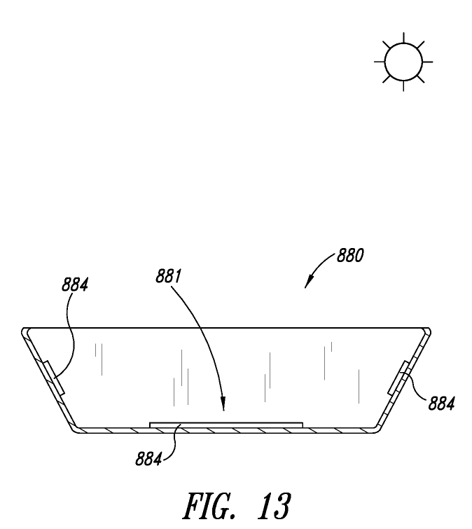

Various types of lighting systems can be employed with the bioreactors, such as the bioreactors 800, 840, 880. For example, FIG. 13 shows an open bioreactor 880 with an auxiliary system 881 configured to actively affect biomass production. The illustrated auxiliary system 881 includes auxiliary production devices 884 spaced apart from one another. In contrast to passive bioreactors that utilize primarily natural resources, output from the auxiliary system 881 can be used to significantly adjust the production of the biomass. Each of the auxiliary production devices 884 can be the same or different and can include one or more light sources (e.g., illumination assemblies 10, light-emitting substrates, waveguides, solar collectors, sensors, and other types of illumination systems), fluid delivery systems for delivering liquids and/or gases, drainage systems, control system, agitators (e.g., horizontal agitators suitable for mixing biomass producing material disposed in horizontally oriented containers), and the like. For example, the auxiliary system 881, in some embodiments, includes one or more light sources that can controllably direct light to the biomass producing material.

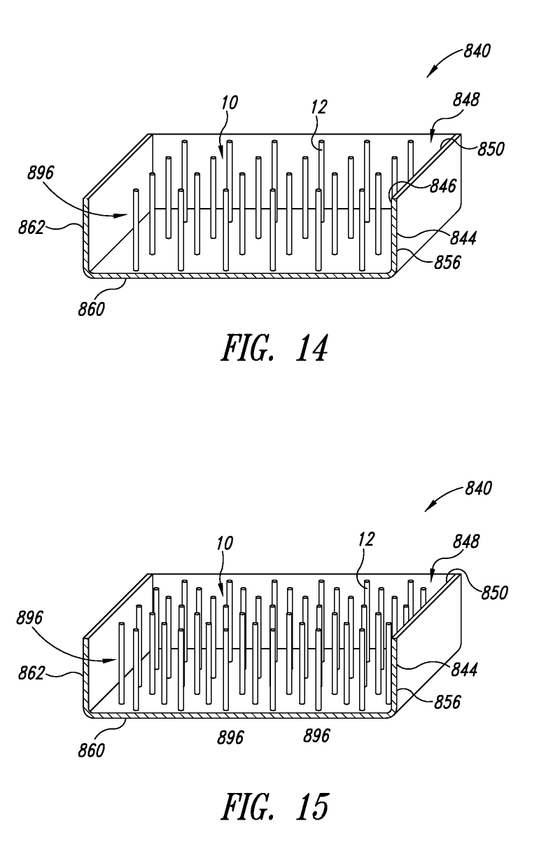

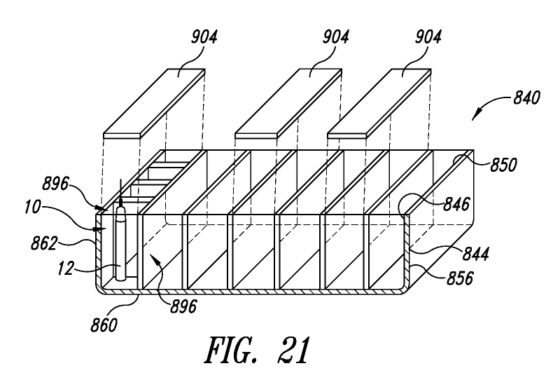

Additionally, various features, components, systems, and sub-systems described herein with respect to closed bioreactors can be incorporated into open bioreactors. For example, referring to FIGS. 14 through 21, in some embodiments, the open bioreactor 800, 840, 880 may include an illumination system 896 comprising one or more of the previously described illumination assemblies 10 each including at least one optical waveguide 12. The one or more illumination assemblies 10 are configured to supply light to at least some of a plurality of photosynthetic organisms retained in the reservoir 810, 848. The one or more illumination assemblies 834 may take the form of a plurality of optical waveguides 12 having an outer surface 22 that forms part of a light energy supplying area.

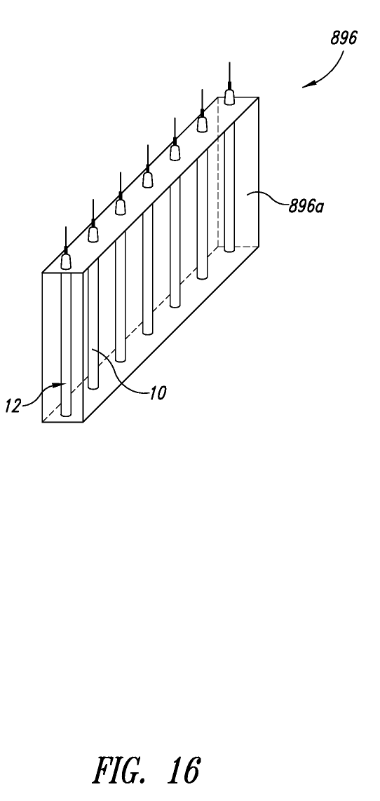

The one or more illumination assemblies 10 may be carried, suspended, or provided by permanent, semi-permanent, and/or removably affixed structures. In some embodiments, the one or more illumination assemblies 10 may be received within the reservoir 810, 848 and substantially held in place by, and/or suspended from, for example, floating booms, floating dry docks, and the like. In some embodiments, as shown in FIG. 16, the illumination system 8 can comprise one or more illumination assemblies 10 that form a single structure illumination system 896a. In some embodiments, multiple single structure illumination system 896a can be received within any of the disclosed bioreactors.

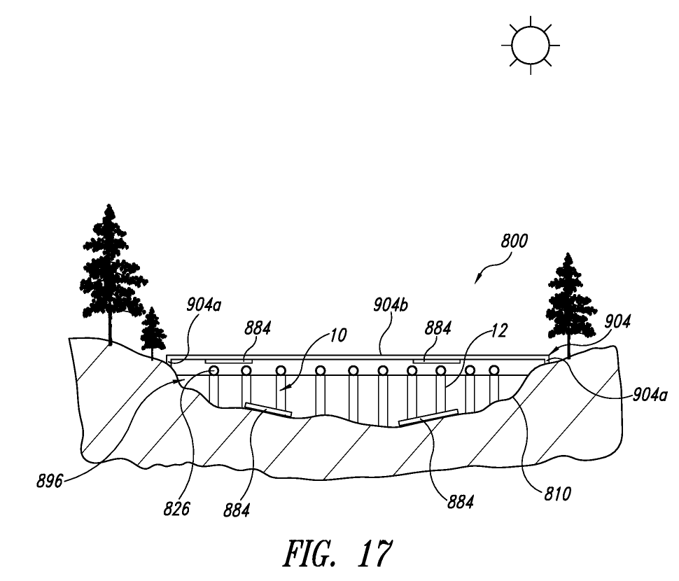

Referring to FIG. 17, in some embodiments, bioreactors 800 including reservoirs 810 such as natural reservoirs (e.g., a lake, pond, stream, canal, or other naturally occurring body of water) may be adapted to create a closed-system, a substantially closed-system, a partially closed-system, or variations thereof adapted for biomass production. In some embodiments, a reservoir 810 may be adapted to create a controlled environment system adapted for biomass production. For example, the reservoir 810 can be in the form of a covered canal with a controlled environment within the enclosed space.

As previously noted, biomasses such as, for example, algal biomasses are often cultured in open-air systems (e.g., ponds, raceway ponds, lakes, natural reservoirs, artificial reservoirs, and the like, as well as regular and irregular shaped structures capable of sustaining biomass growth) that are subject to contamination, or are limited by the inability to substantially control the various process parameters (e.g., temperature, incident light intensity, flow, pressure, nutrients, and the like) involved in cultivating algae. Accordingly, some embodiments include systems, devices, and methods for environmental control of biomass production in open-air systems.

In some embodiments, for example, the bioreactor 800 may include an isolator 904 configured to partially isolate, substantially isolate, completely isolate, or variations thereof the reservoir 810 from a surrounding open air environment. The illustrated isolator 904 can include supports 904a and cover 904b extending between the supports 904a. The cover 904b extends above and across the biomass in the reservoir 810. Along with the isolator 904, the bioreactor 800 can include an illumination system 896 comprising one or more of the previously described illumination assemblies 10. The one or more illumination assemblies 10 may be received within the reservoir 810 and substantially held in place or suspended by structures 826. Examples of structures 826 include floating booms, floating dry docks, and the like.

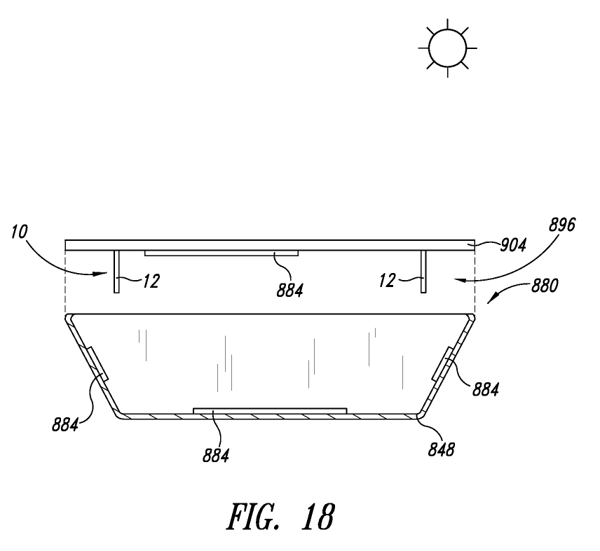

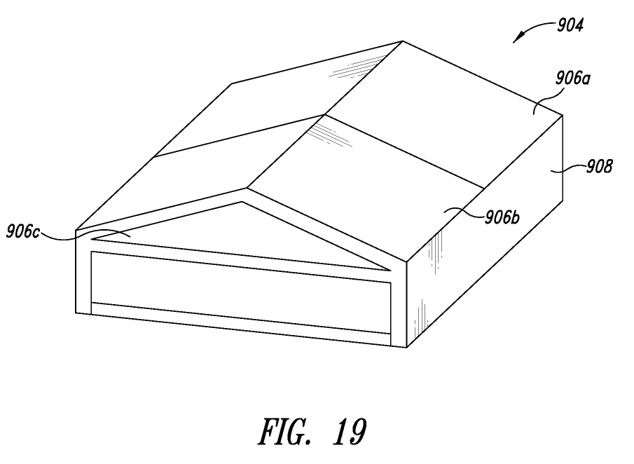

Referring to FIG. 18, in some embodiments, an open bioreactor 880 may be adapted to create a closed-system, a substantially closed-system, a partially closed-system, or variations thereof adapted for biomass production. For example, the bioreactor 880 may be adapted to include an isolator 904 configured to partially isolate, substantially isolate, completely isolate, or variations thereof the bioreactor 880 from a surrounding open air environment. In some embodiments, bioreactor 880 may be adapted to create a controlled environment system adapted for biomass production. In some embodiments, bioreactor 880 may include auxiliary production devices 884 spaced from one another. In contrast to passive bioreactors that utilize primarily natural resources, output from the auxiliary production devices 884 can be used to significantly adjust the production of the biomass. In some embodiments, one or more of the previously described illumination assemblies 10 may be carried by the isolator 904 and adapted to provide sufficient light to sustain dense populations of photosynthetic organisms cultivated within the bioreactor 880.

Referring to FIG. 19, the isolator 904 may take any regular or irregular shape, and may have a cross-section of any suitable geometric form. The isolator 904 may be constructed of any suitable materials. The illustrated isolator 904 includes one or more panels 906a and cover 906b extending between supports 908. The isolator 904 can also include other support structures 906c configured to extend above and/or across a biomass in the bioreactor.

In some embodiments, the isolator 904 is configured to control one or more process parameters (e.g., temperature, incident light intensity, flow, pressure, nutrients, and the like) involved in cultivating algae. For example, the isolator 904 may include one or more structures, coatings, filters, operatives, masks, shades, panels, levers, or combinations thereof for controlling the amount of light (natural or artificial) passing through the isolator 904 and onto a biomass retained in a bioreactor. In some embodiments, the panels 906a, 906b may comprise an optical material (e.g., transparent, translucent, or light-transmitting material, and the like) suitable to permit the passage of artificial or natural into the bioreactor.

In some embodiments, portions 906a, 906b, 906c, 908 of the isolator 904 may be configured to control the duration that the light operates on portions of, for example, an algal mass in the bioreactor, cycling the light (to include periods of light and dark), for example artificial light, to extend the growth of the algae past daylight hours, controlling the wavelength of the light, controlling the lighting patterns, and/or controlling the intensity of the light. For example, the panels 906a, 906b may be moved to adjust the amount of light, if any, that reaches the biomass. The supports 906c, 908 may further include vertical panels that can be moved to adjust the amount of light, if any, that reaches the biomass.

FIGS. 17, 18, 20, and 21 show various open bioreactors 840, 880 that have been modified to include one or more environment controlling structures 904. The one or more environment controlling structures 904 may be operable to partial isolate, substantially isolate, completely isolate, or variations thereof the various open bioreactors 840, 880 from an open air environment. As previously disclosed, one or more illumination assemblies 10 can be carried by the isolator 904 and adapted to provide sufficient light to sustain dense populations of photosynthetic organisms cultivated within the bioreactors 840, 880.

In some embodiments, the one or more environment controlling structures 904 may be configured to control one or more process parameters (e.g., temperature, incident light intensity, flow, pressure, nutrients, and the like) involved in cultivating algae. In some embodiments, the one or more environment controlling structures 904 may be configured to limit access of the biomass retained in the various open bioreactors 840, 880 from the outside.

Some open bioreactors 840, 880 may be limited in their ability to provide sufficient light to sustain dense populations of photosynthetic organisms cultivated within. Accordingly, in some embodiments, the environment controlling structures 904 may include one or more auxiliary production devices 884 carried by the structure 904. For example, the auxiliary production devices 884 may be carried by various components of the structure 904, such as panels 906a, 906b and/or the support structures 906c, and 908. As previously noted, in some embodiments, the one or more auxiliary production devices 884 may take the form of any of the disclosed light-emitting substrates suitable to provide a sufficient amount of light to sustain dense populations of photosynthetic organisms cultivated within the bioreactors 840, 880.

In some embodiments, the environment controlling structures 904 may be optically coupled to a source of solar energy and/or optically coupled to a portion of the one or more auxiliary production devices 884 received within. The source of solar energy may include a solar collector 910 and a solar concentrator 912 optically coupled to the solar collector and a portion of at least one of the auxiliary production devices 884. The solar concentrator can be configured to concentrated solar energy provided by the solar collector and to provide the concentrated solar energy to one or more auxiliary production devices 884.

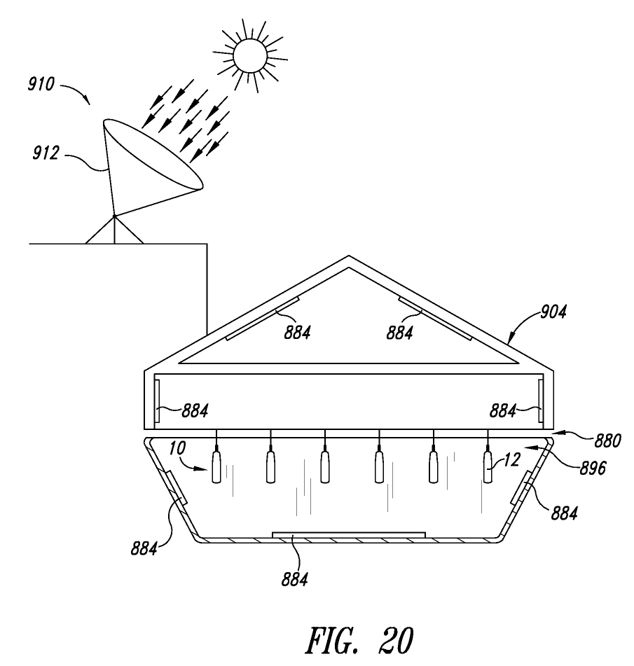

As illustrated in FIG. 20, the bioreactors 840 can be modified to include one or more environment controlling structures 904.

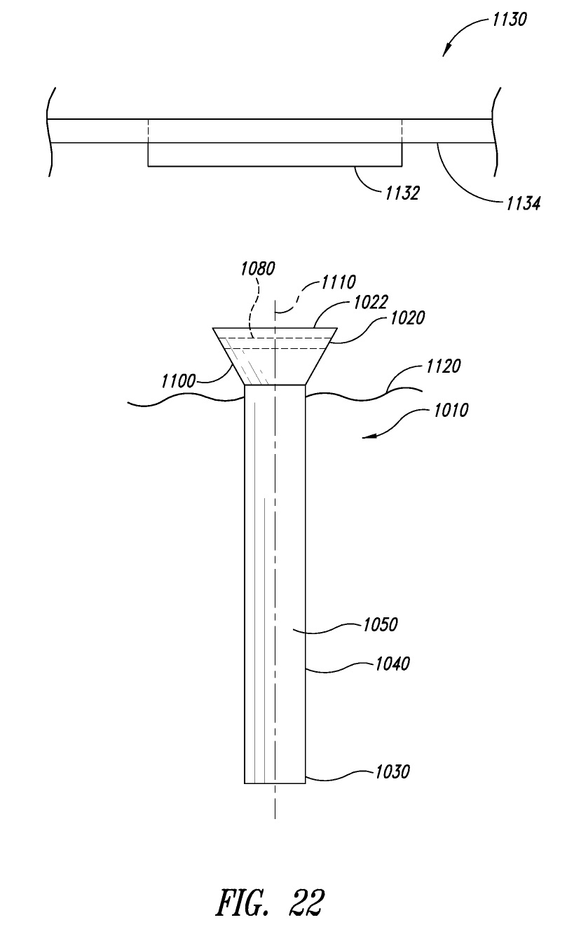

A wide range of different types of optical waveguides can be incorporated into the bioreactors disclosed herein. FIG. 22 illustrates an optical member 1010 in the form of a light-diffusing rod adapted to receive and output diffused light energy. The light-diffusing rod 1010 is generally similar to the optical waveguides described above, except as detailed below.

The light-diffusing rod 1010 of FIG. 22 includes an energy collector end 1020, a terminal end 1030, and a main body 1040 extending between the ends 1020, 1030. The main body 1040 can be substantially optically transparent and forms an outer surface 1050. The main body 1040 is adapted to transmit light energy collected by the energy collector end 1020 such that the light energy is emitted from the outer surface 1050 and/or the terminal end 1030.

The energy collector end 1020 includes one or more integral solar energy collectors. Various types of solar energy collectors may be permanently or temporarily integrated into the solar collector end 1020. In some embodiments, a solar energy collector 1080 (shown in phantom line) is embedded within material forming main body 1040. For example, the solar energy collector 1080 can be in the form of the solar energy collector 104 as discussed in connection with FIG. 5. In other embodiments, the solar energy collector 1080 is physically coupled to an external surface 1100 of the energy collector end 1020. In yet other embodiments, the solar energy collector 1080 can be spaced apart from the rod 1010. For example, a bracket or other mounting component can hold the solar energy collector 1080 above the rod 1010 such that the solar energy collector 1080 directs solar energy into the upper end of the rod 1010.

The collector end 1020, in some embodiments, extends outwardly with respect to a longitudinal axis 1110 of the rod 1010. The illustrated collector end 1020 extends outwardly beyond at least a portion of or the entire outer surface 1050 of the main body 1040. The solar energy collector 1080 may include a lens (such as a Fresnel lens) mounted to a mirrored-surfaced funnel-shaped collector. In operation, the collector end 1020 can be positioned above biomass 1120 such that light energy received by the solar collector end 1020 is transmitted along the main body 1040 and ultimately into the biomass 1120 in which the rod 1010 is submerged.

The solar collector end 1020 can have a generally V-shaped profile, U-shaped profile, spherical configuration, flat configuration, frusto-conical (e.g., funnel-shaped), or any other suitable configuration for providing a relatively large surface area for absorbing light energy when illuminated. By way of example, the rod 1010 can be incorporated into the bioreactor of FIG. 17 such that a relatively large amount of light energy passing through the isolator 904 can be conveniently received by the energy collector end 1020 protruding upwardly from the biomass. Light energy received by an upper surface 1022 of the energy collector end 1020 is transmitted by the rod 1010 to the biomass. In such embodiments, the isolator 904 can include various optical components, such as transparent panels, lenses, mirrors, and the like, that direct solar energy towards the solar energy collector end 1020. Additionally or alternatively, one or more optical fibers can connect the rods 1010 to a separate solar collector(s) or other light sources.

The dimensions of the rod 1010 can be selected based on the desired amount of energy to be delivered into the biomass 1120. In some embodiments, the rod 1010 has a transverse cross-sectional area (i.e., the cross-sectional area taken perpendicularly to the longitudinal axis 1110 of the rod 1010) of at least about 1 cm², 10 cm², 20 cm², 50 cm², 100 cm², 500 cm² and ranges encompassing such cross-sectional areas. Other cross-sectional areas are also possible, if needed or desired.

With continued reference to FIG. 22, a covering 1130 can direct solar light energy to the rod 1010. The covering 1130 includes an optical component 1132 for concentrating solar light energy and delivering the concentrated solar light energy to the rod 1010. The optical component 1132 can be one or more lenses, transparent panels, and the like. In some embodiments, the optical component 1132 is fixedly coupled to a frame 1134 of the covering 1130 to maintain a desired spatial relationship between the optical component 1132 and the rod 1010. In this manner, the optical component 1132 can direct light energy through the air and into the rod 1010. Of course, the optical component 1132 may include different types of auxiliary systems, such as those described above. By way of example, the covering 1130 can be a cover for a canal or other reservoir and the optical component 1132 can be a lens optically coupled to the rod 1010 via air, one or more optical fibers, or both.

The light-diffusing member 1010 can also be in the form of one or more plates, sheets, sheaths, fibers, panels, and the like, as well other types of waveguides with a wide range of shapes. One or more portions of the member 1010 can be partially or fully opaque and may have a monolayer and multilayer construction. The light-diffusing member 1010 can be a hollow structure ora solid structure.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety, including but not limited to: U.S. Pat. No. 5,581,447 and U.S. Pat. No. 5,637,207, are incorporated herein by reference, in their entirety.

Aspects of the various embodiments can be modified, if necessary, to employ systems, circuits, and concepts of the various patents, applications, and publications to provide yet further embodiments, including those patents and applications identified herein. While some embodiments may include all of the light systems, reservoirs, containers, and other structures discussed above, other embodiments may omit some of the light systems, reservoirs, containers, or other structures. Still other embodiments may employ additional ones of the light systems, reservoirs, containers, and structures generally described above. Even further embodiments may omit some of the light systems, reservoirs, containers, and structures described above while employing additional ones of the light systems, reservoirs, containers generally described above.

As one of skill in the art would readily appreciate, the present disclosure comprises systems, devices and methods incorporating light sources to cultivate and/or grow biomasses, photosynthetic organisms, living cells, biological active substances, and the like, by any of the systems, devices and/or methods described herein.

These and other changes can be made in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. An illumination system, comprising:
a substantially optically transparent waveguide having a first end, a second end, an interior, and an outer surface;
a solar energy collector operable to collect a first amount of light energy and optically coupled to the waveguide;
a plurality of light sources located proximate the first end of the waveguide, the plurality of light sources operable to supply a second amount of light energy;
one or more structures proximate the first end of the waveguide, the one or more structures configured to direct light energy comprising at least one of the first amount of light energy from the solar energy collector and the second amount of light energy from the plurality of light sources along said interior of the waveguide; and
a plurality of light-diffusing structures located along the outer surface of the waveguide, the plurality of light-diffusing structures configured to guide at least a portion of the light energy that is directed by the one or more structures along the interior of the waveguide to an exterior of the waveguide.

2. The illumination system of claim 1 wherein the one or more structures are configured to direct only the first amount of light energy from the solar energy collector when the plurality of light sources are OFF.

3. The illumination system of claim 1 wherein the one or more structures are configured to direct only the second amount of light energy from the plurality of light sources when the solar energy collector is OFF.

4. The illumination system of claim 1 wherein said plurality of light sources include light-emitting diodes.

5. The illumination system of claim 1 wherein the waveguide is cylindrical.

6. The illumination system of claim 1, further comprising:
at least one optical fiber extending from the first end of the waveguide to the solar energy collector.

7. The illumination system of claim 1 wherein the waveguide comprises a light-transmitting material.

8. The illumination system of claim 1 wherein the substantially optically transparent cylindrical waveguide comprises at least one material selected from acetal copolymers, acrylic, glass, thermoplastic polymers, thermoset polymers, acrylonitrile butadaine styrene polymers, cellulosic, epoxy, ethylene butyl acrylate, ethylene tetrafluoroethylene, ethylene vinyl alcohol, fluorinated ethylene propylene, furan, nylon, phenolic, poly[2,2,4-trifluoro-5-trifluoromethoxy-1,3-dioxole-co-tetrafluoroethylene], poly[2,2-b]strifluoromethyl-4,5-difluoro-1,3-dioxole-co-tetrafluoroethylene], poly[2,3-(perfluoroalkenyl)perfluorotetrahydrofuran], polyacrylonitrile butadiene styrene, polybenzimidazole, polycarbonate, polyester, polyetheretherketone, polyetherimide, polyethersulfone, polyethylene, polyimide, polymethyl methacrylate, polynorbornene, polyperfluoroalkoxyethylene, polystyrene, polysulfone, polyurethane, polyvinyl chloride, polyvinylidene fluoride, such as diallyl phthalate, thermoplastic elastomer, thermoset polyester, transparent polymers, vinyl ester, or combinations thereof.

9. The illumination system of claim 1 wherein the waveguide comprises an acrylic rod.

10. The illumination system of claim 1, further comprising:
a light filtering system operable to selectively limit a transmission of infrared and ultraviolet solar energy from the solar energy collector to the waveguide.

11. The illumination system of claim 10, wherein the solar energy collector includes the light filtering system.

12. The illumination system of claim 10 wherein the plurality of light-diffusing structures on the outer surface of the waveguide are arranged such that the first and the second amounts of light directed along the interior of the waveguide are guided to the exterior to provide substantially uniform illumination throughout a surface of the waveguide.

13. The illumination system of claim 1 wherein the plurality of light-diffusing structures include one or more etchings, facets, grooves, or combinations thereof.

14. The illumination system of claim 1, further comprising:
a controller operable to control at least one of a light intensity, an illumination intensity, a light-emitting pattern, a peak emission wavelength, an on-pulse duration, or a pulse frequency, or combinations thereof, of the first amount of light energy, the second amount of light energy, or both.

15. The illumination system of claim 1 wherein the plurality of light sources are angularly spaced with respect to one another about an axis of the waveguide.

16. The illumination system of claim 1 wherein the illumination system is operable to provide a photon flux of about 100 micromoles per square meter per second to about 1400 micromoles per square meter per second.

17. The illumination system of claim 1 wherein the illumination system is operable to provide a photon flux of about 200 micromoles per square meter per second to about 600 micromoles per square meter per second.

18. The illumination system of claim 1 wherein the plurality of light sources are operable to provide:
at least one peak emission wavelength ranging from about 440 nm to about 660 nm;
an on-pulse duration ranging from about 1 μs to about 10 s; and
a pulse frequency ranging from about 1 μs to about 10 s.

19. The illumination system of claim 1 wherein the solar energy collector includes a solar concentrator assembly.

20. A bioreactor system for cultivating photosynthetic organisms, comprising:
a container having an exterior surface and an interior surface, the interior surface defining an isolated space configured to retain a plurality of photosynthetic organisms and a cultivation media; and
an illumination assembly coupled to the container and including:
at least one substantially optically transparent waveguide received in the isolated space of the container, the at least one substantially optically transparent waveguide having a first end, a second end, an interior, and an outer surface;
a solar energy collector optically coupleable to at least one waveguide and operable to supply a first amount of light energy;
a plurality of light sources located proximate the first end of the at least waveguide, the plurality of light sources operable to supply a second amount of light energy;
a plurality of structures proximate the first end of the at least one waveguide, the plurality of structures configured to direct energy comprising at least one of the first amount of light energy from the solar energy collector and the second amount of light energy from the plurality of light sources along said interior of the at least one waveguide; and
a plurality of light-diffusing structures located along the outer surface of the at least one waveguide, the plurality of light-diffusing structures configured to guide at least a portion of the energy that is directed by the plurality of structures along said interior of the at least one waveguide to the exterior of the at least one waveguide.

21. The bioreactor system of claim 20, further comprising:
a plurality of photosynthetic organisms, wherein the at least one waveguide is configured to supply an effective amount of light to a substantial portion of the plurality of photosynthetic organisms retained in the isolated space.

22. The bioreactor system of claim 20, further comprising:
a plurality of photosynthetic organisms, wherein the illumination assembly is operable to provide a photon flux of about 100 micromoles per square meter per second to about 1400 micromoles per square meter per second to a substantial portion of the plurality of photosynthetic organisms retained in the isolated space.

23. The bioreactor system of claim 20 wherein the illumination assembly is configured to provide a sufficient amount of light to sustain a biomass concentration having an optical density (OD) value greater than from about 0.1 grams/liter to about 17.5 grams/liter.

24. The bioreactor system of claim 20 wherein the at least one waveguide is configured to provide an amount of light including one or more peak emissions associated with an absorption spectra of either or both chlorophyll a and chlorophyll b.

25. The bioreactor system of claim 20 wherein the plurality of light sources are operable to provide:
a first peak emission wavelength ranging from about 430 nm to about 460 nm;
a second peak emission wavelength ranging from about 660 nm to about 650 nm;
optionally a third peak emission wavelength ranging from about 500 nm to about 570 nm;
an on-pulse duration ranging from about 1 μs to about 10 s; and
a pulse frequency ranging from about 1 μs to about 10 s.

26. The bioreactor system of claim 20 wherein the plurality of light sources include at least one light-emitting diode array.

27. The bioreactor system of claim 20 wherein the illumination assembly includes a plurality of optical waveguides to optically couple a source of light located externally with respect to the container to the at least one waveguide received in the isolated space of the container.

28. The bioreactor system of claim 20 wherein the illumination system further comprises:
at least one optical waveguide on the exterior surface of the container optically coupled to optically couple a source of solar energy to the at least one waveguide received in the isolated space of the container.

29. The bioreactor system of claim 20 wherein the solar energy collector further comprises:
a solar concentrator optically coupled to the solar collector to provide concentrated solar energy from the solar collector to the at least one waveguide received in the isolated space of the container.

30. The bioreactor system of claim 20, further comprising:
one or more sensors operable to detect at least one of a temperature, a pressure, a light intensity, an optical density, a gas content, a pH, a fluid level, or a sparging gas flow rate; and
a controller configured to control at least one of an illumination intensity, an illumination pattern, a peak emission wavelength, an on-pulse duration, and a pulse frequency based on the sensed at least one of the temperature, the pressure, the light intensity, the optical density, the gas content, the pH, the fluid level, or the sparging gas flow rate.

31. The bioreactor system of claim 20, further comprising:
a plurality of photosynthetic organisms, wherein the plurality of photosynthetic organisms are selected from a group comprising prokaryotic algae and eukaryotic algae.

32. The bioreactor system of claim 20, further comprising:
a plurality of photosynthetic organisms, wherein the plurality of photosynthetic organisms are selected from one or more micro-algae.

33. An illumination assembly, comprising:
a waveguide having a first end, a second end, an interior, and an outer surface, the first end of the waveguide adapted to receive a first amount of light energy;
at least one light source located proximate the first end of the waveguide, the at least one light source operable to supply a second amount of light energy;
at least one structure proximate the first end of the waveguide, the at least one structure configured to direct energy comprising at least one of the first amount of light energy and the second amount of light energy along said interior of the waveguide; and
at least one light-diffusing structure located along the outer surface of the waveguide, the at least one optical diffusing structure configured to guide at least a portion of the energy that is directed by the at least one structure along the interior of the waveguide to an exterior of the waveguide.

34. The illumination assembly of claim 33 wherein the at least one light source includes a light-emitting diode.

35. The illumination assembly of claim 33 wherein the at least one structure includes a reflective coating, a reflective material, a mirror structure, a lens structure, or combinations thereof.

36. The illumination assembly of claim 33 wherein the at least one light-diffusing structure includes an etched structure, a facet, a groove, or combinations thereof.

37. The illumination assembly of claim 33 wherein the first end of the waveguide is adapted to receive a first amount of light energy from an optical fiber optically coupled to a solar energy collector.

38. A biomass reactor comprising:
a biomass containment region adapted to contain biomass; and
an illumination system adapted to illuminate the biomass in the biomass containment region, the illumination system comprising a plurality of light-diffusing members spaced apart from one another and at least partially submerged in the biomass, each light-diffusing member adapted to receive light energy and to output the light energy towards the biomass along a length of each member.

39. The biomass reactor of claim 38, further comprising:
a solar energy delivery system adapted to receive solar light energy and to direct that solar light energy to at least one of the light-diffusing members.

40. The biomass reactor of claim 39 wherein the solar energy delivery system includes a solar energy collector and an optical element, the optical element optically coupling the solar energy collector to at least one of the light-diffusing members.

41. The biomass reactor of claim 40 wherein the optical element is optically coupled to all of the light-diffusing members.

42. The biomass reactor of claim 40, further comprising:
a control system adapted to control an amount of solar light energy directed through the optical element to the at least one of the light-diffusing members.

43. The biomass reactor of claim 39 wherein the solar energy delivery system includes a solar concentrator assembly for concentrating solar light energy and delivering the concentrated solar light energy to the plurality of light-diffusing members.

44. The biomass reactor of claim 39, further comprising:
a covering positioned above at least a portion of the biomass containment region, the covering carrying at least a portion of the solar energy delivery system, and wherein the biomass containment region is a reservoir.

45. The biomass reactor of claim 44 wherein the reservoir is a lake, a pond, or a canal.

46. The biomass reactor of claim 38, further comprising:
an energizable light source optically coupled to at least one of the plurality of light-diffusing members, the light source adapted to receive electrical energy and to output the light energy.

47. The biomass reactor of claim 46 wherein each of the light-diffusing members includes a first end, a second end, and an outer surface between the first end and the second end, the first end adapted to receive light energy from an array of light emitting elements of the light source.

48. The biomass reactor of claim 38 wherein each of the light-diffusing members is a rod that includes one or more light-diffusing structures.

49. The biomass reactor of claim 38, further comprising:
a passive light energy system optically coupled to the plurality of light-diffusing members, the passive light energy system adapted to receive solar light energy and to direct the solar light energy to the plurality of light-diffusing members; and
an activatable auxiliary system optically coupled to the plurality of light-diffusing members, the activatable auxiliary system operable to produce non-solar light energy and to direct the non-solar light energy to the plurality of light-diffusing members.

50. The biomass reactor of claim 38 wherein at least one of the light-diffusing members has an enlarged solar energy collector end.

51. An illumination system for biomass production, the system comprising:
a plurality of light-diffusing members;
a passive light energy system optically coupled to the plurality of light-diffusing members, the passive light energy system adapted to receive solar light energy and to direct the solar light energy to the plurality of light-diffusing members; and
an activatable auxiliary system optically coupled to the plurality of light-diffusing members, the activatable auxiliary system adapted to receive electrical energy and to generate non-solar light energy for delivery to the plurality of light-diffusing members.

52. The illumination system of claim 51, further comprising:
a controller configured to control the activatable auxiliary system based, at least in part, on an amount of solar light energy directed to the plurality of light-diffusing members.

53. The illumination system of claim 52 wherein the controller is further configured to cause the activatable auxiliary system to generate non-solar light energy when the passive light energy system directs less than a desired amount of solar light energy to the plurality of light-diffusing members.

54. The illumination system of claim 51 wherein the activatable auxiliary system is adapted to operate independently of the passive light energy system.

55. The illumination system of claim 51 wherein the passive light energy system includes a solar energy collector operable to collect solar light energy and an optical transmission element optically coupled between the solar energy collector and at least one of the light-diffusion members.

56. The illumination system of claim 51 wherein the activatable auxiliary system is configured to generate the non-solar light energy when an amount of solar light energy delivered to the plurality of light-diffusing members falls below a light energy threshold.

57. The illumination system of claim 51 wherein the activatable auxiliary system includes light sources physically coupled to respective ones of the light-diffusing members.

58. An elongate light-diffusing member, comprising:
a solar energy collector end;
a terminal end opposing the solar energy collector end, and
a substantially optically transparent main body extending between the solar energy collector end and the terminal end, the transparent main body having an outer surface such that light energy collected by the solar energy collector end is transmitted through the main body towards the terminal end and is emitted from the outer surface.

59. The elongate light-diffusing member of claim 58 wherein the solar energy collector end includes an integral solar energy collector.

60. The elongate light-diffusing member of claim 58 wherein the solar energy collector end extends outwardly away from a longitudinal axis of the member and beyond at least a portion of the outer surface.

61. The elongate light-diffusing member of claim 60 wherein the solar energy collector end and the terminal end are monolithically formed with the main body.
